SEQUENCE LISTING (70 sequences)

SEQ ID NO: 1 - protein, 353 aa, Artificial Sequence
  Synthetic Construct
ASLTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - DNA, 1059 bp, Artificial Sequence
  Synthetic Construct
gcaagcttgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgtcgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 3 - protein, 106 aa, Artificial Sequence
  Synthetic Construct
TAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 318 bp, Artificial Sequence
  Synthetic Construct
actgcggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 5 - protein, 123 aa, Artificial Sequence
  Synthetic Construct
QEQLVESGGGVVQPGRSLRLSCEASGFIFSAFVLHWVRQAPGKGLEWVAVVWYDGNSKYYADSVKGRFTISRDTSQNTLHLQMDSLRPEDTAVYYCAREWVADDDTFDGFDVWGQGTMVTVSS

SEQ ID NO: 6 - DNA, 369 bp, Artificial Sequence
  Synthetic Construct
caggagcagctggtggagtctgggggaggagtggtccagcctgggaggtccctgagactctcctgtgaagcgtctggtttcatcttcagtgcctttgtcttgcactgggtccgacaggctccaggcaaggggctggagtgggtggcagttgtttggtatgatggaaatagtaaatactatgcagactccgtgaagggccgattcaccatctccagagacacttcccagaacacactgcatctgcaaatggacagcctgcgtcccgaggacacggctgtctattactgtgcgagagaatgggtggcggacgatgatacttttgatggttttgatgtctggggccaagggacaatggtcaccgtctcttca

SEQ ID NO: 7 - protein, 110 aa, Artificial Sequence
  Synthetic Construct
EIVLSQSPATLSLSPGERATLSCRASQSVTNSLAWYQQKPGQAPRLLIYDASNRATGIPARFSGSGSGTDFTLTISSLEPEDFAVYYCQQRSNWPPEVTFGPGTKVDIKR

SEQ ID NO: 8 - DNA, 330 bp, Artificial Sequence
  Synthetic Construct
gaaattgtgttgtcacagtctccagccaccctgtctttgtctccaggggaaagagccaccctctcctgcagggccagtcagagtgttaccaactccttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggaggtcactttcggccctgggaccaaagtggatatcaaacga

SEQ ID NO: 9 - protein, 8 aa, Artificial Sequence
  Synthetic Construct
GFIFSAFV

SEQ ID NO: 10 - DNA, 24 bp, Artificial Sequence
  Synthetic Construct
ggtttcatcttcagtgcctttgtc

SEQ ID NO: 11 - protein, 8 aa, Artificial Sequence
  Synthetic Construct
VWYDGNSK

SEQ ID NO: 12 - DNA, 24 bp, Artificial Sequence
  Synthetic Construct
gtttggtatgatggaaatagtaaa

SEQ ID NO: 13 - protein, 16 aa, Artificial Sequence
  Synthetic Construct
AREWVADDDTFDGFDV

SEQ ID NO: 14 - DNA, 48 bp, Artificial Sequence
  Synthetic Construct
gcgagagaatgggtggcggacgatgatacttttgatggttttgatgtc

SEQ ID NO: 15 - protein, 6 aa, Artificial Sequence
  Synthetic Construct
QSVTNS

SEQ ID NO: 16 - DNA, 18 bp, Artificial Sequence
  Synthetic Construct
cagagtgttaccaactcc

SEQ ID NO: 17 - protein, 3 aa, Artificial Sequence
  Synthetic Construct
DAS

SEQ ID NO: 18 - DNA, 9 bp, Artificial Sequence
  Synthetic Construct
gatgcatcc

SEQ ID NO: 19 - protein, 11 aa, Artificial Sequence
  Synthetic Construct
QQRSNWPPEVT

SEQ ID NO: 20 - DNA, 33 bp, Artificial Sequence
  Synthetic Construct
cagcagcgtagcaactggcctccggaggtcact

SEQ ID NO: 21 - protein, 128 aa, Artificial Sequence
  Synthetic Construct
QVQLVQSGAEVKKPGSSVKVSCKASGDTFIRYSFTWVRQAPGQGLEWMGRIITILDVTHYAPHLQGRVTITADKFTSTVYLELRNLRSDDTAVYFCAGVYRGRGGRGEYDNNGFLKHWGQGTLVTVSS

SEQ ID NO: 22 - DNA, 384 bp, Artificial Sequence
  Synthetic Construct
caggtgcagctggtgcagtctggggctgaggtgaagaagcctgggtcctcggtgaaggtctcctgcaaggcctctggagacaccttcatcagatatagttttacctgggtgcgacaggcccctggacaaggccttgagtggatgggaaggatcatcactatccttgatgtaacacactacgcaccgcaccttcagggcagagtcacgattaccgcggacaagttcacgagcacagtctacctggagctgcggaatctaagatctgacgatacggccgtatatttctgtgcgggagtgtaccgagggagaggcggacgaggggaatatgataataatgggtttctgaaacattggggccagggaaccctggtcaccgtctcctca

SEQ ID NO: 23 - protein, 107 aa, Artificial Sequence
  Synthetic Construct
EMTQSPATLSVSPGERATLSCRASESVSSDLAWYQQKPGQAPRLLIYGASTRATGVPARFSGSGSGAEFTLTISSLQSEDFAVYYCQQYNNWPPRYTFGQGTRLEIK

SEQ ID NO: 24 - DNA, 322 bp, Artificial Sequence
  Synthetic Construct
gagatgacccagtctccagccaccctgtctgtgtctccaggggaaagagccaccctctcctgcagggccagtgagagtgttagtagcgacttagcctggtaccagcagaaacctggccaggctcccaggctcctcatatatggtgcatccaccagggccaccggtgtcccagccaggttcagtggcagtgggtctggggcagagttcactctcaccatcagcagcctgcagtctgaagattttgcagtttattactgtcagcagtacaataactggcctccgaggtacacttttggccaggggaccaggctggagatcaaac

SEQ ID NO: 25 - protein, 8 aa, Artificial Sequence
  Synthetic Construct
GDTFIRYS

SEQ ID NO: 26 - DNA, 24 bp, Artificial Sequence
  Synthetic Construct
ggagacaccttcatcagatatagt

SEQ ID NO: 27 - protein, 8 aa, Artificial Sequence
  Synthetic Construct
IITILDVT

SEQ ID NO: 28 - DNA, 24 bp, Artificial Sequence
  Synthetic Construct
atcatcactatccttgatgtaaca

SEQ ID NO: 29 - protein, 21 aa, Artificial Sequence
  Synthetic Construct
AGVYRGRGGRGEYDNNGFLKH

SEQ ID NO: 30 - DNA, 63 bp, Artificial Sequence
  Synthetic Construct
gcgggagtgtaccgagggagaggcggacgaggggaatatgataataatgggtttctgaaacat

SEQ ID NO: 31 - protein, 6 aa, Artificial Sequence
  Synthetic Construct
ESVSSD

SEQ ID NO: 32 - DNA, 18 bp, Artificial Sequence
  Synthetic Construct
gagagtgttagtagcgac

SEQ ID NO: 33 - protein, 3 aa, Artificial Sequence
  Synthetic Construct
GAS

SEQ ID NO: 34 - DNA, 9 bp, Artificial Sequence
  Synthetic Construct
ggtgcatcc

SEQ ID NO: 35 - protein, 11 aa, Artificial Sequence
  Synthetic Construct
QQYNNWPPRYT

SEQ ID NO: 36 - DNA, 33 bp, Artificial Sequence
  Synthetic Construct
cagcagtacaataactggcctccgaggtacact

SEQ ID NO: 37 - protein, 119 aa, Artificial Sequence
  Synthetic Construct
EVQLVQSGAEVKKPGATVKISCKASGYTFSDFYMYWVRQAPGKGLEWMGLIDPEDADTMYAEKFRGRVTITADTSTDTGYLELSSLRSEDTAVYYCAADPWELNAFNVWGQGTLVSVSS

SEQ ID NO: 38 - DNA, 357 bp, Artificial Sequence
  Synthetic Construct
gaggtccagctggtacagtccggggctgaggtgaagaagcctggggccaccgtgaagatctcctgcaaggcttctggatacaccttcagcgacttctacatgtactgggtgcgacaggcccctgggaaagggcttgagtggatgggccttattgatcctgaagatgctgatacaatgtatgcagagaagttccggggcagagtcaccataaccgcagacacgtctacagacacaggctatttggaactgagcagcctgagatctgaggacacggccgtctattactgtgcagcagatccgtgggaactaaatgcatttaatgtctggggccaagggacactggtcagcgtctcttca

SEQ ID NO: 39 - protein, 106 aa, Artificial Sequence
  Synthetic Construct
DIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIK

SEQ ID NO: 40 - DNA, 318 bp, Artificial Sequence
  Synthetic Construct
gacatccagatgacccagtctccatcttctgtgtctgcatctgtaggagacagagtcaccatcacttgtcgggcgagtcaggatattagcacctggttagcctggtatcagcagaaaccagggaaagcccctaagctcctgatatatgctgcatccactttgcaaagtggtgtcccgtcaaggttcagcggcagtggatctgggacagatttcagtctcactatcaacagcctgcagcctgaggattttgcgacttactattgtcaacaggctaacagtttcttcacattcggcggagggaccaaggtggagatcaaa

SEQ ID NO: 41 - protein, 8 aa, Artificial Sequence
  Synthetic Construct
GYTFSDFY

SEQ ID NO: 42 - DNA, 24 bp, Artificial Sequence
  Synthetic Construct
ggatacaccttcagcgacttctac

SEQ ID NO: 43 - protein, 8 aa, Artificial Sequence
  Synthetic Construct
IDPEDADT

SEQ ID NO: 44 - DNA, 24 bp, Artificial Sequence
  Synthetic Construct
attgatcctgaagatgctgataca

SEQ ID NO: 45 - protein, 12 aa, Artificial Sequence
  Synthetic Construct
AADPWELNAFNV

SEQ ID NO: 46 - DNA, 36 bp, Artificial Sequence
  Synthetic Construct
gcagcagatccgtgggaactaaatgcatttaatgtc

SEQ ID NO: 47 - protein, 6 aa, Artificial Sequence
  Synthetic Construct
QDISTW

SEQ ID NO: 48 - DNA, 18 bp, Artificial Sequence
  Synthetic Construct
caggatattagcacctgg

SEQ ID NO: 49 - protein, 3 aa, Artificial Sequence
  Synthetic Construct
AAS

SEQ ID NO: 50 - DNA, 9 bp, Artificial Sequence
  Synthetic Construct
gctgcatcc

SEQ ID NO: 51 - protein, 8 aa, Artificial Sequence
  Synthetic Construct
QQANSFFT

SEQ ID NO: 52 - DNA, 24 bp, Artificial Sequence
  Synthetic Construct
caacaggctaacagtttcttcaca

SEQ ID NO: 53 - protein, 134 aa, Artificial Sequence
  Synthetic Construct
QVRLVQSGAEVNKPGSSVKVSCQASGATLNSHAFSWVRQAPGQGLEWMAGIIPIFGSSHYAQKFQGRVTITADESTRTVYLHLRGLRSDDTAVYYCASNSIAGVAAAGDYADYDGGYYYDMDVWGQGTTVTVSS

SEQ ID NO: 54 - DNA, 402 bp, Artificial Sequence
  Synthetic Construct
caggtgcggctggtgcagtctggggctgaggtgaacaagcctgggtcctcggtgaaggtctcctgtcaggcttctggagccaccttaaacagtcatgctttcagctgggtgcgacaggcccctggacaagggcttgagtggatggcagggatcatccctatatttggttcatcacactacgcacaaaagttccagggcagagtcacgattaccgcggacgagtccacgcgcacagtgtatttgcatctgagaggcctgagatctgatgacacggccgtttattactgtgcgagtaattctattgccggagtagcagctgccggagactacgctgactacgatgggggctactactacgatatggatgtctggggccaagggaccacggtcaccgtctcctca

SEQ ID NO: 55 - protein, 110 aa, Artificial Sequence
  Synthetic Construct
QSILTQPPSVSTAPGQKVTISCSGSSSNIGNNDVSWYQQFPGTVPKLVIYENNQRPSGIPDRFSGSKSGTSATLGITGLQTGDEADYYCGTWDSSLSAVVFGGGSKVTVL

SEQ ID NO: 56 - DNA, 331 bp, Artificial Sequence
  Synthetic Construct
cagtctattttgacgcagccgccctcagtgtctacggccccaggacagaaggtcaccatctcctgctctggaagcagctccaacattgggaataatgatgtttcctggtaccagcagttcccaggaacagtccccaaactcgtcatctatgaaaataatcagcgaccctcagggattcctgaccgcttctctggctccaagtccggcacgtcagccaccctgggcatcaccggactccagactggggacgaggccgattattactgcggcacatgggatagcagcctgagtgctgtggtcttcggcggagggtccaaggtgaccgtcctag

SEQ ID NO: 57 - protein, 8 aa, Artificial Sequence
  Synthetic Construct
GATLNSHA

SEQ ID NO: 58 - DNA, 24 bp, Artificial Sequence
  Synthetic Construct
ggagccaccttaaacagtcatgct

SEQ ID NO: 59 - protein, 8 aa, Artificial Sequence
  Synthetic Construct
IIPIFGSS

SEQ ID NO: 60 - DNA, 24 bp, Artificial Sequence
  Synthetic Construct
atcatccctatatttggttcatca

SEQ ID NO: 61 - protein, 27 aa, Artificial Sequence
  Synthetic Construct
ASNSIAGVAAAGDYADYDGGYYYDMDV

SEQ ID NO: 62 - DNA, 81 bp, Artificial Sequence
  Synthetic Construct
gcgagtaattctattgccggagtagcagctgccggagactacgctgactacgatgggggctactactacgatatggatgtc

SEQ ID NO: 63 - protein, 8 aa, Artificial Sequence
  Synthetic Construct
SSNIGNND

SEQ ID NO: 64 - DNA, 24 bp, Artificial Sequence
  Synthetic Construct
agctccaacattgggaataatgat

SEQ ID NO: 65 - protein, 3 aa, Artificial Sequence
  Synthetic Construct
ENN

SEQ ID NO: 66 - DNA, 9 bp, Artificial Sequence
  Synthetic Construct
gaaaataat

SEQ ID NO: 67 - protein, 11 aa, Artificial Sequence
  Synthetic Construct
GTWDSSLSAVV

SEQ ID NO: 68 - DNA, 33 bp, Artificial Sequence
  Synthetic Construct
ggcacatgggatagcagcctgagtgctgtggtc

SEQ ID NO: 69 - protein, 16 aa, Artificial Sequence
  Synthetic Construct
DGFDVWGQGTMVTVSS

SEQ ID NO: 70 - protein, 11 aa, Artificial Sequence
  Synthetic Construct
TFGPGTKVDIK

(19) United States
(12) Patent Application Publication
(10) Pub. No.: US 2015/0175678 A1
(43) Pub. Date: Jun. 25, 2015

(54) IgA CD4i ANTIBODIES AND METHODS OF TREATMENT USING SAME

(71) Applicant: Beth Israel Deaconess Medical Center, Inc., Boston, MA (US)

(72) Inventors: Lisa Cavacini, Natick, MA (US); Marshall Posner, Mount Vernon, NY (US); Xiaocong Yu, Needham, MA (US)

(21) Appl. No.: 14/408,182

(22) PCT Filed: Jun. 17, 2013
(86) PCT No.: PCT/US13/46124

§ 371 Date: Dec. 15, 2014

Related U.S. Application Data
(60) Provisional application No. 61/660,541, filed on Jun. 15, 2012.
(60) Provisional application No. 61/665,536, filed on Jun. 28, 2012.

(51) Int. Cl. C07K 16/10; A61K 47/48; A61K 39/42

(52) U.S. Cl. CPC C07K 16/1063; A61K 39/42; A61K 47/48561; C07K 2317/522; C07K 2317/524; C07K 2317/526; C07K 2317/565; C07K 2317/24; C07K 2317/21; A61K 2039/505

(57) ABSTRACT

The present invention relates to isolated IgA antibodies, or fragments thereof, which have variable domains derived from an antibody that specifically binds to a CD4-induced (CD4i) epitope. In particular, the isolated IgA antibodies display enhanced neutralization activity relative to their IgG, non-chimeric counterparts. The invention also provides methods for therapy with the isolated IgA antibodies for the treatment of a subject having a viral infection or having an increased risk of a viral infection.

### CROSS-REFERENCE TO RELATED APPLICATION

This application claims the benefit of the filing date of U.S. Provisional Application No. 61/660,541, filed Jun. 15, 2012, and U.S. Provisional Application No. 61/665,536, filed Jun. 28, 2012, both of which are hereby incorporated by reference in their entirety.

### STATEMENT AS TO FEDERALLY FUNDED RESEARCH

This invention was made with government support under Grant Nos. AI075932 and AI063986, awarded by the National Institutes of Health (NIH). The government has certain rights in the invention.

### BACKGROUND OF THE INVENTION

Notwithstanding the success of drug treatment in controlling an established human immunodeficiency virus (HIV) infection, additional means of therapy are required for more effective, long-term control and for prevention. Whereas the humoral immune response plays a significant role in the control of a number of human viral diseases, numerous studies have clearly demonstrated the general inability of the humoral immune system to develop functionally effective neutralizing antibodies during natural infection or vaccination. The immune system is confounded by the immunogenicity of the variable loops which are exposed on the surface of the virus and which tend to elicit strain specific antibodies as well the transient exposure of specific neutralizing epitopes upon virion binding or engagement with cluster of differentiation 4 (CD4). Regardless, neutralizing antibodies can be captured and are effective at preventing infection in several non-human primate models.

Structural analysis of a number of neutralizing antibodies suggests that even if immunogens can be designed to express neutralizing epitopes, it may be difficult to induce the effective antibody structures needed to robustly neutralize the virus. The practical application of passive immunotherapy is also currently severely limited by the quantities of antibodies required for systemic therapeutic levels and timing. Thus, there remains a need for novel interventions to prevent and treat HIV infection.

### SUMMARY OF THE INVENTION

We have found that IgA isotype-switched antibody variants of CD4-induced (CD4i) IgG antibodies display significant neutralizing activities compared to the parental hybrid or any IgG isotype counterparts in the absence of soluble CD4 (sCD4). We have also discovered that the CD4i-specific IgA antibodies of the invention possess significantly increased antibody-dependent cell-mediated virus inhibition (ADCVI) of certain human immunodeficiency virus (HIV) clades and HIV-infected cells compared to their IgG isotype counterparts.

Accordingly, the invention features CD4i-specific IgA antibodies and methods for treating subjects (e.g., humans) having a viral infection (e.g., an HIV infection) or prophylactically treating subjects (e.g., humans) having an increased risk of a viral infection (e.g., an HIV infection), where such methods include a CD4i-specific IgA antibody or fragment thereof.

In a first aspect, the invention features an isolated IgA antibody, or fragment thereof (e.g., having antigen binding activity), wherein the variable domains of the IgA antibody are derived from an antibody that specifically binds to a CD4i epitope of a polypeptide (e.g., F425-A1g8, 17b, 48d, E51, X5, or m16). In one embodiment, the IgA antibody, or fragment thereof, includes at least one heavy chain constant domain of IgA selected from the group consisting of a CH1, CH2, and CH3 constant domain of IgA. In another embodiment, the IgA antibody, or fragment thereof, includes at least two heavy chain constant domains of IgA selected from the group consisting of a CH1, CH2, and CH3 constant domain of IgA (e.g., CH1 and CH2, CH1 and CH3, or CH2 and CH3). In another embodiment the IgA antibody, or fragment thereof, includes three heavy chain constant domains of IgA, where the three heavy chain constant domains are CH1, CH2, and CH3 constant domains of IgA. The IgA antibody may include a heavy chain constant region amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 1. In certain embodiments, the antibody, or fragment thereof, is a chimeric antibody containing IgA constant domains and IgG-derived variable domains.

In some embodiments, the isolated IgA antibody, or fragment thereof, includes a light chain constant domain of IgA. The IgA antibody may include a light chain constant domain amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 3.

In any of the embodiments described herein, the isolated IgA antibody, or fragment thereof, includes variable domains derived from F425-A1g8, 17b, 48d, E51, X5, or m16.

In some embodiments for which the isolated IgA antibody, or fragment thereof, includes variable domains derived from F425-A1g8, the antibody, or fragment thereof, may include a heavy chain variable domain and/or a light chain variable domain, wherein the heavy chain variable domain includes a CDR-H1 including the amino acid sequence GFIFSAFV (SEQ ID NO: 9), CDR-H2 including the amino acid sequence VWYDGNSK (SEQ ID NO: 11), and CDR-H3 including the amino acid sequence AREWVADDDTFDGFDV (SEQ ID NO: 13), and/or wherein the light chain variable domain includes a CDR-L1 including the amino acid sequence QSVTNS (SEQ ID NO: 15), CDR-L2 including the amino acid sequence DAS (SEQ ID NO: 17), and CDR-L3 including the amino acid sequence QQRSNWPPEVT (SEQ ID NO: 19). The above isolated F425-A1g8 IgA antibodies, or fragments thereof, may include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 5 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 7.

In some embodiments for which the isolated IgA antibody, or fragment thereof, includes variable domains derived from 17b, the antibody, or fragment thereof, may include a heavy chain variable domain and/or a light chain variable domain, wherein the heavy chain variable domain includes a CDR-H1 including the amino acid sequence GDTFIRYS (SEQ ID NO: 25), CDR-H2 including the amino acid sequence IITILDVT (SEQ ID NO: 27), and CDR-H3 including the amino acid sequence AGVYRGRGGRGEYDNNGFLKH (SEQ ID NO: 29), and/or wherein the light chain variable domain includes a CDR-L1 including the amino acid sequence ESVSSD (SEQ ID NO: 31), CDR-L2 including the amino acid sequence GAS (SEQ ID NO: 33), and CDR-L3 including the amino acid sequence QQYNNWPPRYT (SEQ ID NO: 35). The above isolated 17b IgA antibodies, or fragments thereof, may include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 21 and/or a light chain variable domain including an amino acid sequence at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 23.

In some embodiments for which the isolated IgA antibody, or fragment thereof, includes variable domains derived from 48d, the antibody, or fragment thereof, may include a heavy chain variable domain and/or a light chain variable domain, wherein the heavy chain variable domain includes a CDR-H1 including the amino acid sequence GYTFSDFY (SEQ ID NO: 41), CDR-H2 including the amino acid sequence IDPEDADT (SEQ ID NO: 43), and CDR-H3 including the amino acid sequence AADPWELNAFNV (SEQ ID NO: 45), and/or wherein the light chain variable domain includes a CDR-L1 including the amino acid sequence QDISTW (SEQ ID NO: 47), CDR-L2 including the amino acid sequence AAS (SEQ ID NO: 49), and CDR-L3 including the amino acid sequence QQANSFFT (SEQ ID NO: 51). The above isolated 48d IgA antibodies, or fragments thereof, may include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 37 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 39.

In some embodiments for which the isolated IgA antibody, or fragment thereof, includes variable domains derived from E51, the antibody, or fragment thereof, may include a heavy chain variable domain and/or a light chain variable domain, wherein the heavy chain variable domain includes a CDR-H1 including the amino acid sequence GATLNSHA (SEQ ID NO: 57), CDR-H2 including the amino acid sequence IIPIFGSS (SEQ ID NO: 59), and CDR-H3 including the amino acid sequence ASNSIAGVAAAGDYADYDGGYYYDMDV (SEQ ID NO: 61), and/or wherein the light chain variable domain includes a CDR-L1 including the amino acid sequence SSNIGNND (SEQ ID NO: 63), CDR-L2 including the amino acid sequence ENN (SEQ ID NO: 65), and CDR-L3 including the amino acid sequence GTWDSSLSAVV (SEQ ID NO: 67). The above isolated E51 IgA antibodies, or fragments thereof, may include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 53 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 55.

In any of the embodiments described herein, the isolated IgA antibodies, or fragments thereof, can be associated with a J chain polypeptide including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 69 or 70.

In any of the embodiments described herein, the isolated IgA antibodies, or fragments thereof, may further include a label or a toxin (e.g., as described herein).

In any of the embodiments described herein, the isolated IgA antibodies, or fragments thereof, may be chimeric, human, humanized, or synthetic.

In a second aspect, the invention features an isolated IgA antibody, or fragment thereof, that competes for CD4i binding with any one of the antibodies disclosed in the first aspect.

In a third aspect, the invention features a method of treating a subject (e.g., a human) having a viral infection (e.g., an HIV infection), the method includes administering a therapeutically effective amount of any of the isolated IgA antibodies disclosed in the first or second aspect to the subject, thereby treating the subject.

In a fourth aspect, the invention features a method of prophylactically treating a subject (e.g., a human) having an increased risk of a viral infection (e.g., an HIV infection), the method includes administering a therapeutically effective amount of any of the isolated IgA antibodies disclosed in the first or second aspect to the subject, thereby treating the subject.

The isolated IgA antibodies and methods of the invention may be used to treat (or prophylactically treat) a wide variety of viral infections. Viral infections may include any viral infection caused by an infective agent of the Retroviridae family. In one embodiment, the infective agent is a lentivirus. In some embodiments, the lentivirus is a human immunodeficiency virus type 1 (HIV-1), human immunodeficiency virus type 2 (HIV-2), simian immunodeficiency virus (SIV), bovine immunodeficiency virus (BIV), feline immunodeficiency virus (FIV), Jembrana disease virus, equine infectious anaemia (EIA), puma lentivirus (PLV), lion lentivirus (LLV), caprine arthritis encephalitis virus (CAEV), or Maedi-Visna virus. In preferred embodiments, the lentivirus is HIV-1 (e.g., clade B or clade C HW-1). In another preferred embodiment, the lentivirus is HIV-2.

In any of the embodiments described herein, the isolated IgA antibody preferably neutralizes the infective agent (e.g., HIV) in the subject. Typically, the subject is a mammal, such as a human.

The isolated IgA antibodies of the present invention and pharmaceutical compositions thereof may be administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularly, mucosally, intrapericardially, intraumbilically, intraocularally, orally, topically, locally, by inhalation, by injection, by infusion, by continuous infusion, by localized perfusion bathing target cells directly, by catheter, by lavage, in cremes, or in lipid compositions. In preferred embodiments, the isolated IgA antibodies of the present invention and pharmaceutical compositions thereof are administered by infusion, by continuous infusion, mucosally, or subcutaneously. The isolated IgA antibody, or fragment thereof, may be optionally administered as a pharmaceutical composition including a pharmaceutically acceptable carrier, such as physiological saline.

In a further aspect, the invention features polynucleotides encoding antibodies of the invention. In additional aspects, the invention features vectors including the polynucleotides of the invention and a host cell including the vectors. In one embodiment, the host cell is a mammalian cell. In a preferred embodiment, the mammalian cell is a CHO cell. In an additional aspect, the invention features a method of producing an isolated IgA CD4i antibody of the invention that includes culturing the host cell that comprises the vector with IgA antibody-encoding polynucleotides in a culture medium. Preferably, the IgA CD4i antibody is recovered from the host cell or the host cell's culture medium.

In any of the embodiments described herein, the half-life of the IgA CD4i antibody is at least 2-fold greater (e.g., 2-, 3-, 4-, 5-, 6-, 7-, 8-, 9-, 10-, 10²-, or 10³-fold or more greater) than that of the comparable IgG CD4i antibody.

In any of the embodiments described herein, the IgA CD4i antibody may have improved binding to the CD4i epitope compared to that of the comparable IgG CD4i antibody, with Kd values at least 2-fold lower (e.g., 2-, 3-, 4-, 5-, 6-, 7-, 8-, 9-, 10-, 10²-, or 10³-fold or more lower) than that of the IgG CD4i antibody.

In any of the embodiments described herein, the increased neutralization activity of the IgA CD4i antibody is improved over the comparable IgG CD4i antibody with IC₅₀ or IC₉₀ values at least 1.5-fold lower (e.g., 1.5-, 2-, 3-, 4-, 5-, 6-, 7-, 8-, 9-, 10-, 10²-, or 10³-fold or more lower), as described herein in Table 2 or 3.

In any of the embodiments described herein, the increased antibody-dependent cell-mediated virus inhibition (ADCVI) activity of the IgA CD4i antibody is improved over the comparable IgG CD4i antibody with IC₅₀ or IC₉₀ values at least 1.5-fold lower (e.g., 1.5-, 2-, 3-, 4-, 5-, 6-, 7-, 8-, 9-, 10-, 10²-, or 10³-fold or more lower), as described herein in Table 2 or 3.

In any of the embodiments described herein, the CD4i epitope may be located on a gp120 polypeptide (e.g., as described herein).

In any of the embodiments described herein, the IgA antibody may be an antibody having one or more IgA1 constant domains.

For any of the polypeptides or polynucleotides described herein (e.g., light chain variable domain, heavy chain variable domain, light chain constant domain, heavy chain constant domains, J chains, fragments, or polynucleotides encoding the polypeptides described herein), the sequences having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to one or more of SEQ ID NOs: 1-8, 21-24, 37-40, 53-56, 69, and 70.

### DEFINITIONS

As used herein, the term “about” means +/−10% of the recited value.

The terms “antibody” and “immunoglobulin (Ig)” are used interchangeably in the broadest sense and include monoclonal antibodies (e.g., full-length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein). An antibody typically comprises both “light chains” and “heavy chains.” The light chains of antibodies (immunoglobulins) from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA1, and IgA2. The heavy chain constant domains that correspond to the different classes of immunoglobulins are called a, 6, ε, γ, and μ, respectively. The subunit structures and three-dimensional configurations of different classes of immunoglobulins are well known.

“Antibody fragments” of “fragments” comprise only a portion of an intact antibody, wherein the portion preferably retains at least one, preferably most or all, of the functions normally associated with that portion when present in an intact antibody. Examples of antibody fragments include Fab, Fab′, F(ab′)₂, and Fv fragments (e.g., single-chain variable fragments (scFv)); diabodies; linear antibodies; single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function, ADCVI function, and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

By “antibody-dependent cell-mediated virus inhibition” or “ADCVI” is meant an antibody function that inhibits virus yield from infected cells in the presence of Fc receptor-bearing effector cells (e.g., neutrophils). The increased ADCVI activity of the IgA CD4i antibody is improved over the comparable IgG CD4i antibody with IC₅₀ or IC₉₀ values at least 1.5-fold lower (e.g., 1.5-, 2-, 3-, 4-, 5-, 6-, 7-, 8-, 9-, 10-, 10²-, or 10³-fold or more lower), as described herein in Table 2 or 3. Similar to ADCVI, “antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FeyRIII only, whereas monocytes express FcγRI, FcγRII, and FcγRIII FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 or Presta U.S. Pat. No. 6,737,056 may be performed. Alternatively, an in vitro ADCVI assay may be used. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Natl. Acad. Sci. USA 95:652-656 (1998).

“Fc receptor” or “FcR” describes a receptor that binds to the Fc region of an antibody. The preferred FcR is a native sequence human FcR. Moreover, a preferred FcR is one which binds an IgA antibody (an alpha receptor) and includes FcαR (CD89). In other embodiments, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Other FcRs, including those to be identified in the future, are encompassed by the term “FcR” herein.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie™ blue or, preferably, silver stain. Isolated antibody includes the antibody in situ within recombinant cells since at least one component of the antibody's natural environment will not be present. Similarly, isolated antibody includes the antibody in medium around recombinant cells. Ordinarily, however, isolated antibody will be prepared by at least one purification step.

As used herein, “variable domain” of an antibody, or fragment thereof, refers to the portions of the light and heavy chains of antibody molecules that include amino acid sequences of complementarity determining regions (CDRs; i.e., CDR-1, CDR-2, and CDR-3), and framework regions (FRs). VH refers to the variable domain of the heavy chain. VL refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “complementarity determining regions” or “CDRs” refers to the amino acid residues of an antibody variable domain the presence of which are necessary for antigen binding. Each variable domain typically has three CDR regions identified as CDR-1, CDR-2 and CDR-3. Each complementarity determining region may comprise amino acid residues from a “complementarity determining region” as defined by Kabat (i.e., about residues 24-34 (CDR-L1), 50-56 (CDR-L2) and 89-97 (CDR-L3) in the light chain variable domain and 31-35 (CDR-H1), 50-65 (CDR-H2) and 95-102 (CDR-H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (CDR-L1), 50-52 (CDR-L2) and 91-96 (CDR-L3) in the light chain variable domain and 26-32 (CDR-H1), 53-55 (CDR-H2) and 96-101 (CDR-H3) in the heavy chain variable domain; Chothia and Lesk, J. Mol. Biol. 196:901-917 (1987)). In some instances, a complementarity determining region can include amino acids from both a CDR region defined according to Kabat and a hypervariable loop.

As used herein, the term “constant domain” of an antibody refers to any domain that is not a variable domain (e.g., CH1, CH2, CH3, and CL domains).

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDR-H1 includes amino acids H26-H35, the heavy chain FR1 residues are at positions 1-25 and the FR2 residues are at positions 36-49. Common structural features among the variable regions of antibodies, or functional fragments thereof, are well known in the art. The DNA sequence encoding a particular antibody can generally be found following well known methods such as those described in Kabat, et al. 2987 Sequence of Proteins of Immunological Interest, U.S. Department of Health and Human Services, Bethesda Md., which is incorporated herein as a reference. In addition, a general method for cloning functional variable regions from antibodies can be found in Chaudhary, V. K., et al., 1990 Proc. Natl. Acad. Sci. USA 87:1066, which is incorporated herein as a reference.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may, at times, be used interchangeably as the plasmid is the most commonly used form of vector.

“Polynucleotide” or “nucleic acid” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and a basic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

By “cluster of differentiation 4” or “CD4” is meant an isolated, soluble, or cell surface-attached glycoprotein that is capable of revealing a CD4-induced (CD4i) epitope on gp120 upon complexation. CD4 includes, for example, human CD4 protein (NCBI RefSeq No. NP_{—}000607.1).

By “CD4-induced” or “CD4i” epitope is meant a highly conserved epitope of gp120 that may be revealed upon binding of gp120 to CD4. The CD4i epitope is located in or near the CD4 co-receptor binding site and is essential for CD4 binding.

By “competes for binding” is meant an antibody that preferentially binds to an epitope (e.g., CD4i) to the extent that it blocks, to some degree, binding of a reference antibody to the epitope (e.g., CD4i). Competitive binding is determined in an assay in which the immunoglobulin under test inhibits specific binding of a reference antibody to a common antigen, such as CD4i. Numerous types of competitive binding assays are known, for example: solid phase direct or indirect radioimmunoassay (RIA), solid phase direct or indirect enzyme immunoassay (EIA), sandwich competition assay (see Stahli et al, Methods in Enzymology 9:242 (1983)); solid phase direct biotin-avidin EIA (see Kirkland et al, J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I¹²⁵ label (see Morel et al, Mol. Immunol. 25(1):7 (1988)); solid phase direct biotin-avidin EIA (Cheung et al., Virology 176:546 (1990)); and direct labeled RIA (Moldenhauer et al, Scand. J. Immunol. 32:77 (1990)). Typically, such an assay involves the use of purified antigen bound to a solid surface or cells bearing either of these, an unlabeled test immunoglobulin, and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75%, 75-80%, 80-85%, 90-95%, or more.

By “neutralizes” is meant to recognize a specific antigen (e.g., gp120, e.g., the CD4i epitope of gp120) and inhibit the effect(s) of the antigen (e.g., gp120, e.g., the CD4i epitope of gp120) in the host subject (e.g., a human). As used herein, the IgA CD4i antibodies of the invention are neutralizing antibodies.

By “pharmaceutical composition” is meant a composition containing a compound described herein formulated with a pharmaceutically acceptable carrier, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable carrier” is meant a carrier which is physiologically acceptable to a treated mammal (e.g., a human) while retaining the therapeutic properties of the compound with which it is administered. One exemplary pharmaceutically acceptable carrier is physiological saline. Other physiologically acceptable carriers and their formulations are known to one skilled in the art and described, for example, in Remington's Pharmaceutical Sciences (18^{th} edition, A. Gennaro, 1990, Mack Publishing Company, Easton, Pa.), incorporated herein by reference.

By “sequence identity” or “sequence similarity” is meant that the identity or similarity between two or more amino acid sequences, or two or more nucleotide sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (such as C:\seq1.txt); -j is set to a file containing the second nucleic acid sequence to be compared (such as C:\seq2.txt); -p is set to blastn; -o is set to any desired file name (such as C:\output.txt); -q is set to -1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q -1 -r 2.

To compare two amino acid sequences, the options of B12seq can be set as follows: -i is set to a file containing the first amino acid sequence to be compared (such as C:\seq1.txt); -j is set to a file containing the second amino acid sequence to be compared (such as C:\seq2.txt); -p is set to blastp; -o is set to any desired file name (such as C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid or nucleotide residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (i.e., 1166÷1554*100=75.0). The length value will always be an integer. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence.

By “subject” is meant a mammal (e.g., a human).

By “specifically binds” is meant the preferential association of a binding moiety (e.g., an antibody or fragment thereof) to a target molecule (e.g., a viral protein, e.g., gp120, e.g., the CD4i epitope of gp120) in a sample (e.g., a biological sample) or in vivo or ex vivo. It is recognized that a certain degree of non-specific interaction may occur between a binding moiety and a non-target molecule. Nevertheless, specific binding may be distinguished as mediated through specific recognition of the target molecule. Specific binding results in a stronger association between the binding moiety (e.g., an antibody or fragment thereof) and, e.g., an antigen (e.g., gp120 of human immunodeficiency virus (HIV)) than between the binding moiety and, e.g., a non-target molecule (e.g., non-viral polypeptide). In one example, the antibody may specifically bind to a CD4-induced (CD4i) epitope of envelope glycoprotein gp120 of HIV. The antibody (e.g., an IgA antibody) may have, e.g., at least 2-fold greater affinity (e.g., 2, 3, 4, 5, 6, 7, 8, 9, 10, 10²-, 10³-, 10⁴-, 10⁵-, 10⁶-, 10⁷-, 10⁸-, 10⁹-, or 10¹⁰-fold greater affinity) to the gp120 protein than to other viral or non-viral polypeptides (e.g., the IgA antibody has at least 2-fold greater affinity to gp120 than a comparable IgG antibody).

By “therapeutically effective amount” is meant an amount of a therapeutic agent (e.g., an isolated IgA CD4i-specific antibody, or fragment thereof, of the invention) that alone, or together with one or more additional (optional) therapeutic agents, effects beneficial or desired results. The therapeutically effective amount depends upon the context in which the therapeutic agent is applied. For example, in the context of administering a composition of an isolated IgA CD4i-specific antibody, or fragment thereof, of the invention, the therapeutically effective amount of the composition is an amount sufficient to achieve an increase in the levels of the IgA antibody, or fragment thereof (e.g., an extracellular level), and/or to achieve a reduction in the level of an infectious virus (e.g., HIV) as compared to the response obtained without administration of the composition, and/or to prevent the propagation of an infectious virus (e.g., HIV) in a subject (e.g., a human) having an increased risk of viral infection. Ideally, a therapeutically effective amount provides a therapeutic effect without causing a substantial cytotoxic effect in the subject. In general, a therapeutically effective amount of a composition administered to a subject (e.g., a human subject) will vary depending upon a number of factors associated with that subject, for example the overall health of the subject, the condition to be treated, or the severity of the condition. A therapeutically effective amount of a composition can be determined by varying the dosage of the product and measuring the resulting therapeutic response.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliation” of a disease, disorder, or condition means that the extent and/or undesirable clinical manifestations of the disease, disorder, or condition are lessened and/or time course of the progression is slowed or lengthened, as compared to the extent or time course in the absence of treatment. By “treating” a subject (e.g., a human) having a viral infection is meant causing a reduction in the number of infectious virus particles (e.g., human immunodeficiency virus (HIV) particles), slowing or inhibiting an increase in the number of infectious virus particles (e.g., HW particles), reducing the likelihood of an initial or subsequent occurrence of a viral infection, or reducing an adverse symptom associated with a disease, disorder, or condition caused by a viral infection (e.g., acquired immunodeficiency syndrome (AIDS)). In a desired embodiment, the percent of an infective agent (or virus particles of an infective agent) surviving treatment is at least 20, 30, 40, 50, 60, 70, 80, 90, or 100% lower than the initial number of infective agents (or virus particles of an infective agent), as measured using any standard assay.

By “prophylactically treating” or “preventing” a disease (e.g., AIDS) or condition (e.g., a viral infection, e.g., an HIV infection) in a subject (e.g., a human) is meant reducing the risk of developing (i.e., the incidence) or reducing the severity of the disease or condition prior to the appearance of symptoms. The prophylactic treatment may completely prevent or reduce appearance of the disease (e.g., AIDS) or a symptom thereof and/or may be therapeutic in terms of a partial or complete cure for a disease (e.g., AIDS) and/or adverse effect attributable to the disease (e.g., AIDS). Prophylactic treatment may include reducing or preventing a disease (e.g., AIDS) or condition (e.g., a viral infection, e.g., an HIV infection) from occurring in an individual who may be predisposed or at an increased risk of developing the disease (e.g., AIDS) or condition (e.g., a viral infection, e.g., an HIV infection) but has not yet been diagnosed as having it.

Other features and advantages of the invention will be apparent from the following Detailed Description and the claims.

### BRIEF DESCRIPTION OF THE DRAWINGS

FIG. 1 shows amino acid and nucleotide sequences of the IgA1 constant domain and light chain constant domain.

FIG. 2A shows the amino acid sequences of the heavy chain variable domain (VH), light chain variable domain (VL), CDR-H1, CDR-H2, CDR-H3, CDR-L1, CDR-L2, and CDR-L3 of the F425-A1g8 antibody.

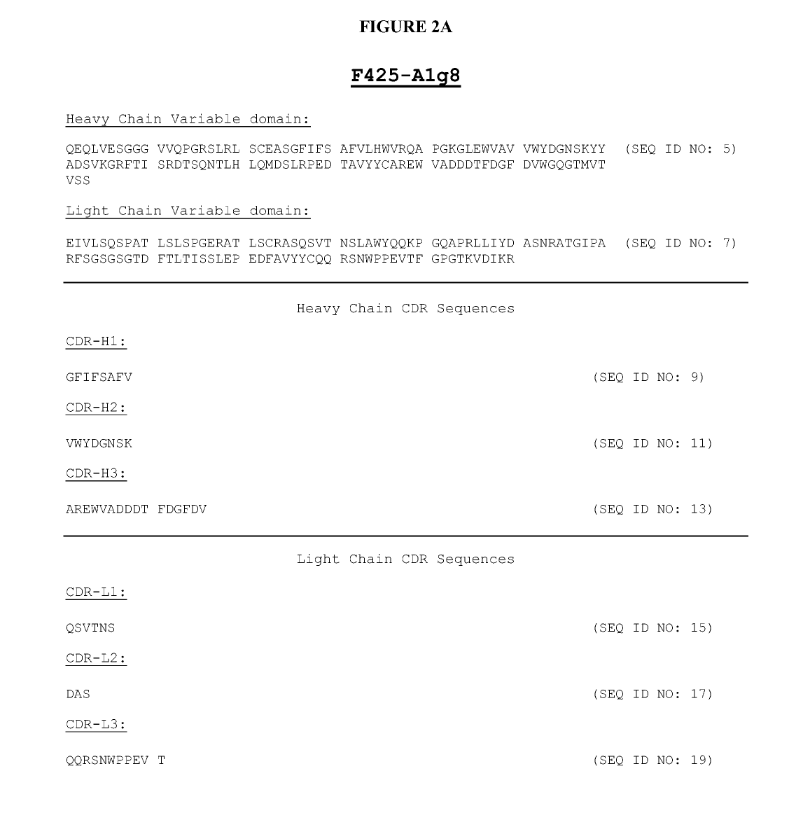

FIG. 2B shows the nucleotide sequences of the heavy chain variable domain (VH), light chain variable domain (VL), CDR-H1, CDR-H2, CDR-H3, CDR-L1, CDR-L2, and CDR-L3 of the F425-A1g8 antibody.

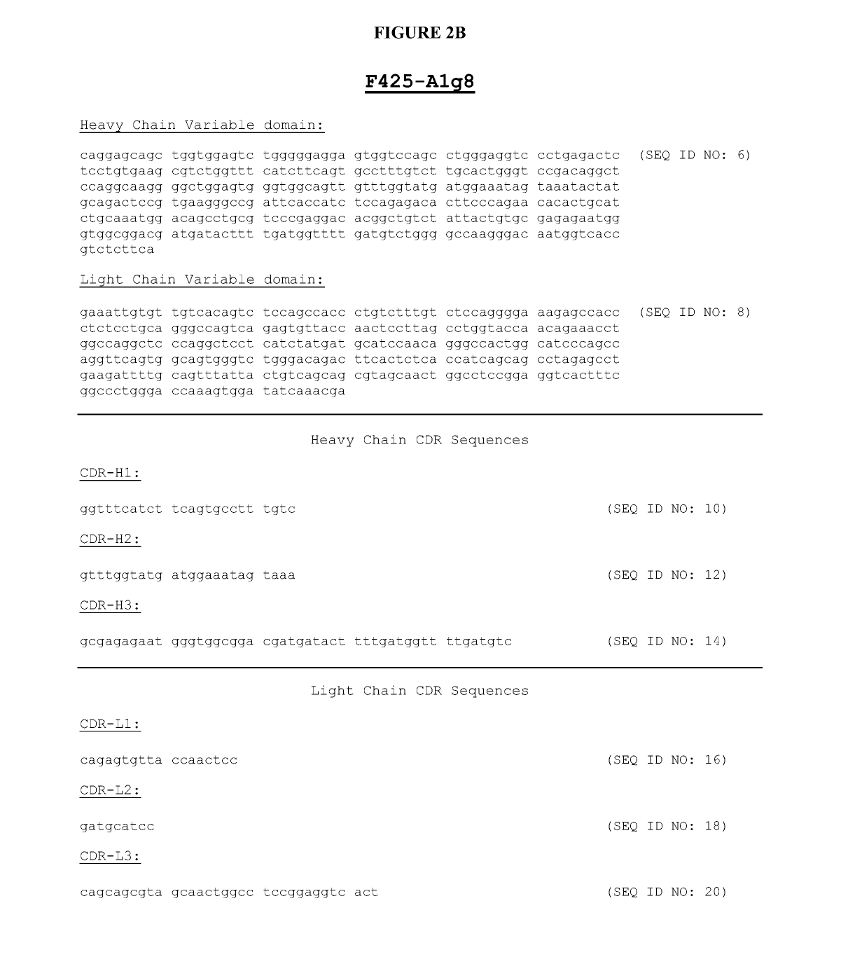

FIG. 3A shows the amino acid sequences of the heavy chain variable domain (VH), light chain variable domain (VL), CDR-H1, CDR-H2, CDR-H3, CDR-L1, CDR-L2, and CDR-L3 of the 17b antibody.

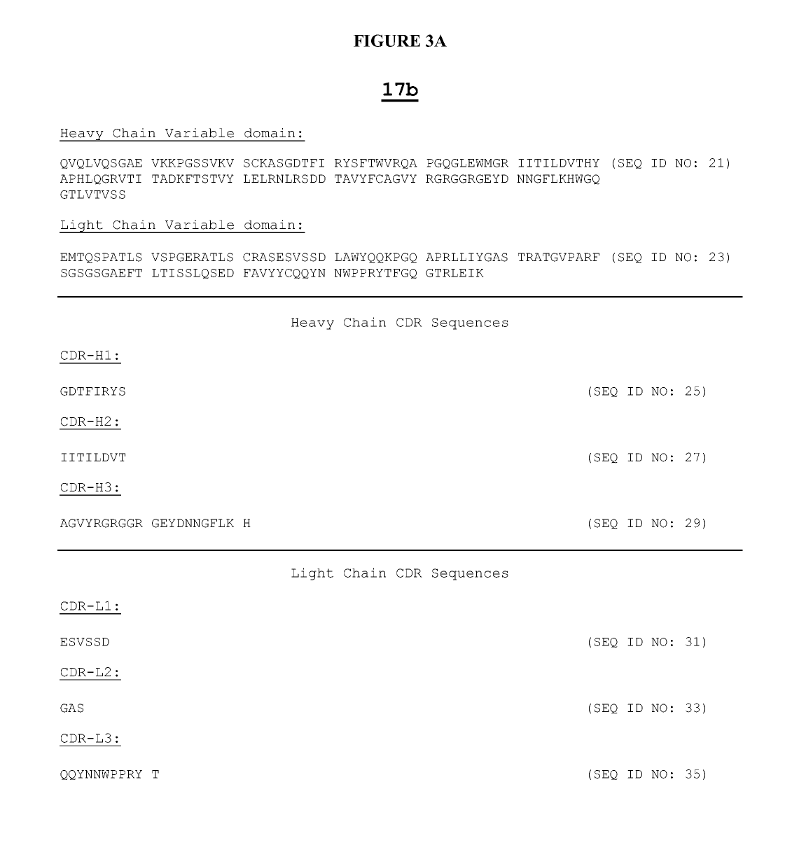

FIG. 3B shows the nucleotide sequences of the heavy chain variable domain (VH), light chain variable domain (VL), CDR-H1, CDR-H2, CDR-H3, CDR-L1, CDR-L2, and CDR-L3 of the 17b antibody.

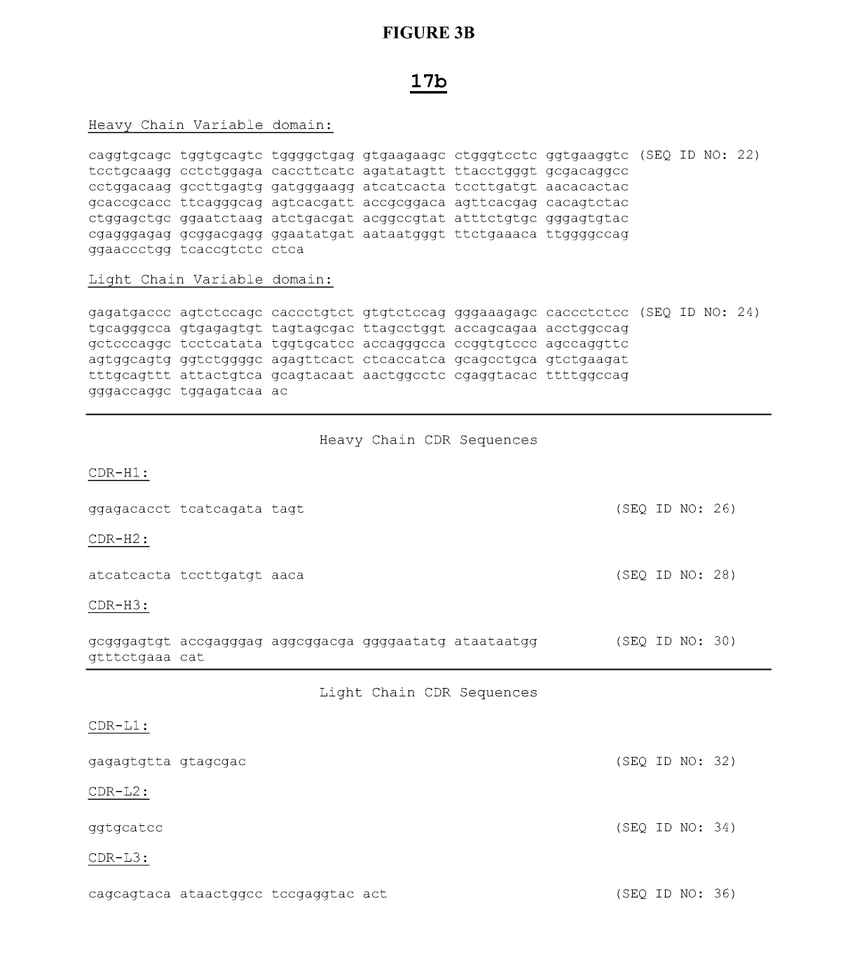

FIG. 4A shows the amino acid sequences of the heavy chain variable domain (VH), light chain variable domain (VL), CDR-H1, CDR-H2, CDR-H3, CDR-L1, CDR-L2, and CDR-L3 of the 48d antibody.

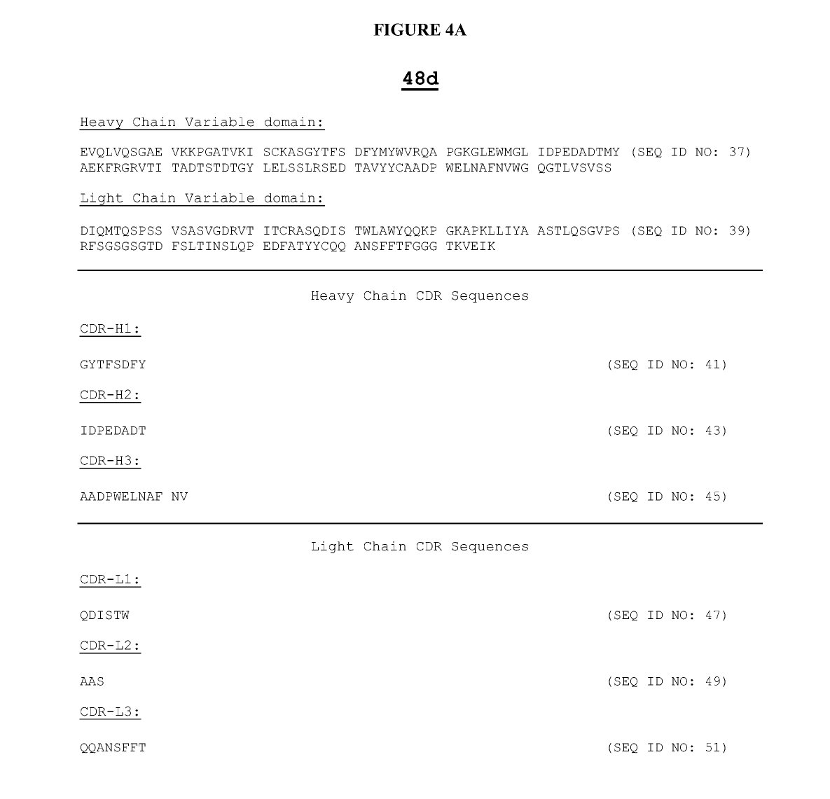

FIG. 4B shows the nucleotide sequences of the heavy chain variable domain (VH), light chain variable domain (VL), CDR-H1, CDR-H2, CDR-H3, CDR-L1, CDR-L2, and CDR-L3 of the 48d antibody.

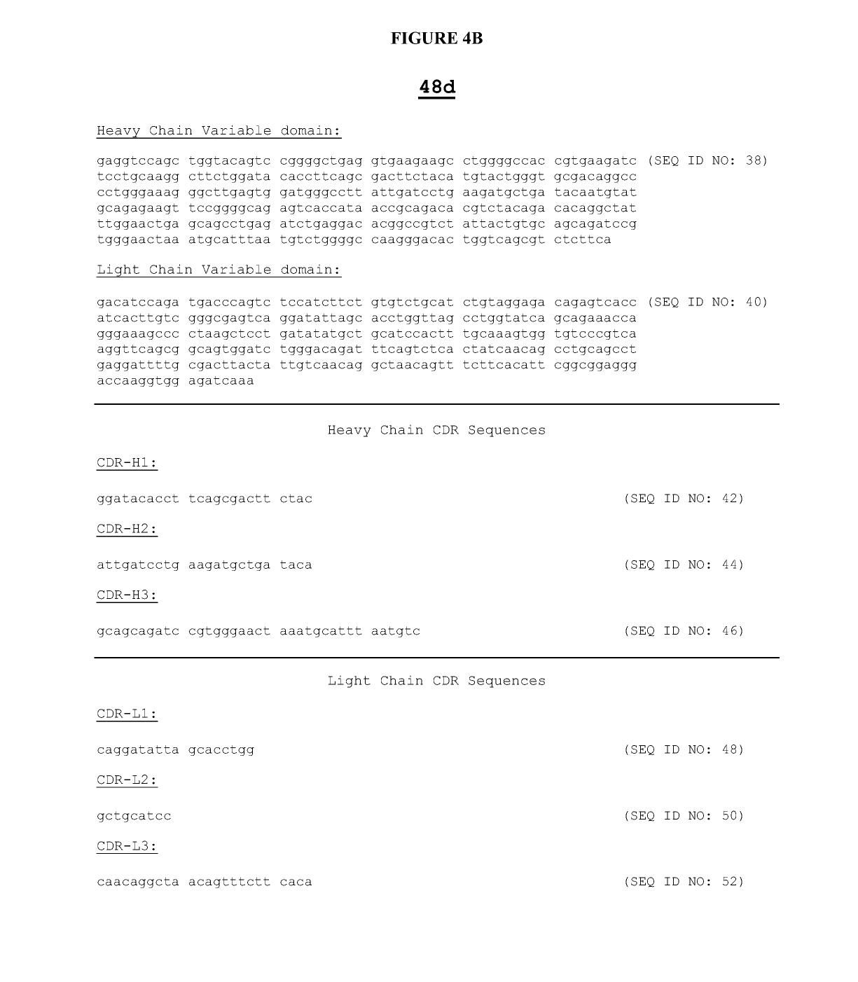

FIG. 5A shows the amino acid sequences of the heavy chain variable domain (VH), light chain variable domain (VL), CDR-H1, CDR-H2, CDR-H3, CDR-L1, CDR-L2, and CDR-L3 of the E51 antibody.

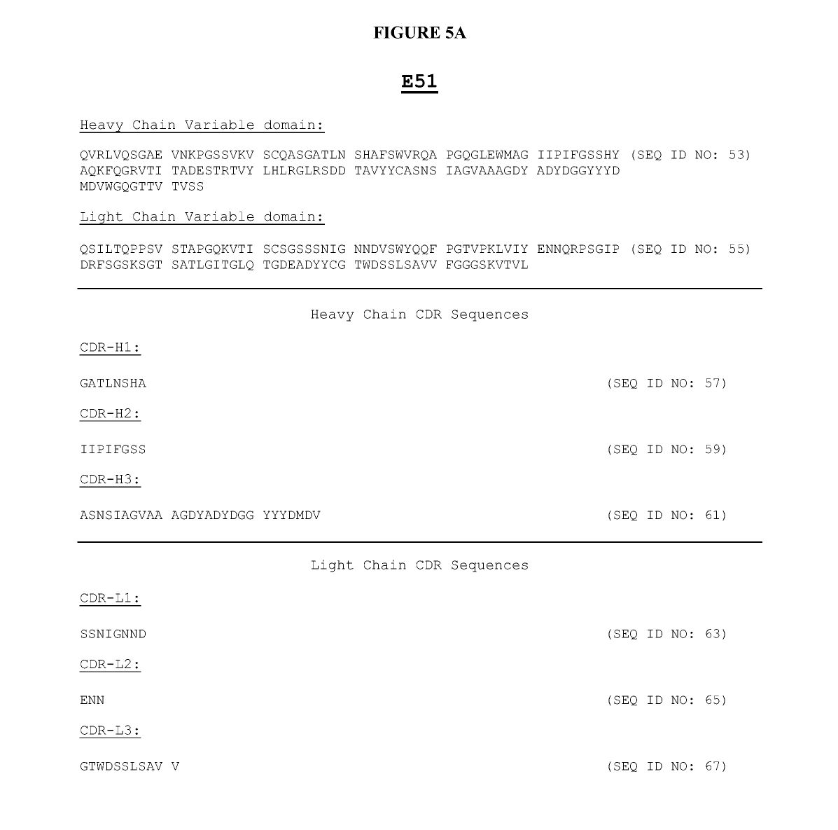

FIG. 5B shows the nucleotide sequences of the heavy chain variable domain (VH), light chain variable domain (VL), CDR-H1, CDR-H2, CDR-H3, CDR-L1, CDR-L2, and CDR-L3 of the E51 antibody.

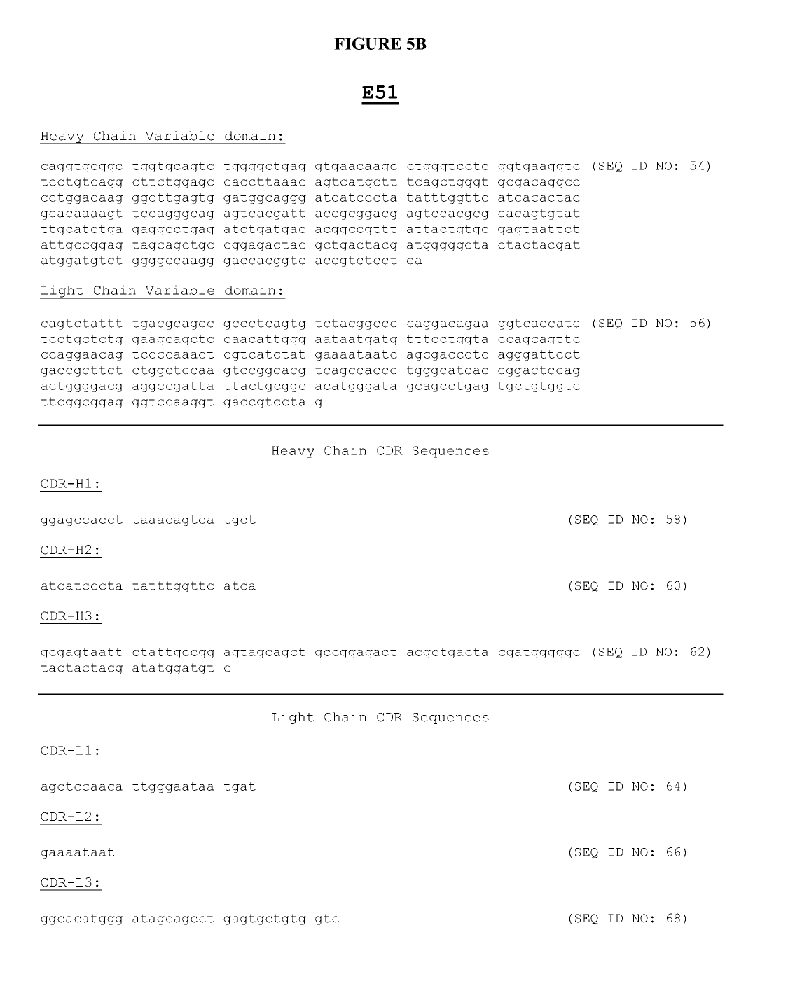

FIG. 6 shows the amino acid sequences of the J chain polypeptide of A1g8 VH (SEQ ID NO: 69) and A1g8 VL (SEQ ID NO: 70).

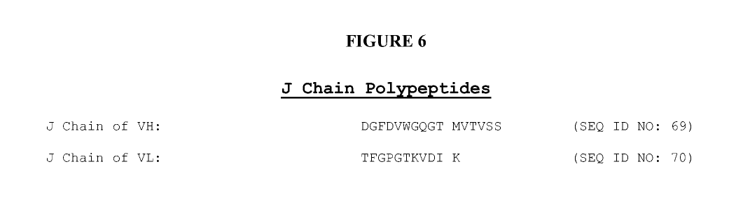

FIG. 7 is a graph showing the immunoreactivity of F425-A1g8 IgA1 and IgG1 antibody variants with HIV-infected cells.

FIG. 8 is a graph showing the neutralization activity of F425-A1g8 IgG1 and IgA1 antibody variants against JR-FL measured using TZM-bl cells.

FIG. 9 is a graph showing antibody-dependent cell-mediated viral inhibition (ADCVI) mediated by F425-A1g8 IgA1 and IgG antibody variants measured using JR-FL-infected peripheral blood mononuclear cells (PBMC).

### DETAILED DESCRIPTION

We generated and characterized isotype switch variants of the CD4-induced (CD4i)-specific antibodies (e.g., F425-A1g8). We discovered that the isolated IgA1 variant of F425-A1g8 displayed significant neutralization activity alone. In contrast, there was little neutralization by the parental hybrid or IgG1 variant in the absence of soluble CD4 (sCD4). Combined with epidemiological data, these data suggest that HIV-specific (e.g., CD4i-specific) IgA antibodies may play an important independent role in providing protective immunity against HIV infection in mucosal surfaces.

The entry of HIV-1 into target cells typically requires the sequential binding of the viral exterior envelope glycoprotein, gp120, to CD4 and a chemokine receptor. The binding of CD4 to gp120 reveals the CD4i epitope. CD4i-specific antibodies (or CD4i antibodies) recognize the epitope of gp120 structures that are formed or exposed by CD4 binding and can block virus binding to the chemokine receptor. However, CD4i neutralizing antibodies demonstrate large conformational requirements for binding in that the site is only exposed upon CD4/gp120 binding which limits antibody access to the proximal chemokine site (Chioe et al., Cell. 114:161-170, 2003). The results of many studies have demonstrated that F(ab) or scFv of CD4i antibodies tend to be more effective at neutralization than the intact molecule, presumably due to greater access to the epitope (Chioe et al., Cell. 114:161-170, 2003; Moulard et al., Proc. Natl. Acad. Sci. USA. 99:6913-6918, 2002). The distinct structural properties of IgA provide this antibody isotype some unique functional capabilities. In some embodiments, IgA1 molecules have a lengthy hinge region with a 16 amino-acid insertion. Crystal studies have shown that the structure of IgA1 resembles more of a “T” structure as compared to the canonical “Y” structure of an IgG1 molecule (Boehm et al., J. Mol. Biol. 286:1421-1447, 1999). We hypothesized that this flexible stretch property of IgA1 would seem likely to afford a greater reach between its two antigen-binding sites and potential to decrease steric hinderance (Broliden et al., Immunol. Lett. 79:29-36, 2001), allowing improved access to the relatively hidden CD4i epitopes recognized by F425-A1g8 compared to IgG1 isotypes. This may be particularly important in an effective neutralizing antibody response to HIV when increasing antibody flexibility could result in cooperative interactions on gp120/gp41 trimers. Increased flexibility of antibody molecules have been shown by our laboratory to increase antibody neutralization activity (Cavacini et al., AIDS Res. Hum. Retroviruses. 19:785-792, 2003).

We also hypothesized that the IgA antibody of the present invention may have particular advantages for inhibiting infectious diseases or for targeting antigens (Bakema et al., mAbs. 3:352-361, 2011; Otten et al., J. Immunol. 174:5472-5480, 2005; Huls et al., Cancer Research. 59:5778-5784, 1999; Dechant et al., Critical Reviews in Oncology/hematology. 39:69-77, 2001; Woof et al., Immunology. 4:89-99, 2004; Ravetch et al., Annu. Rev. Immunol. 19:275-290, 2001). The increase in ADCVI activity observed with the IgA1 construct of F425-A1g8 in our study supports this hypothesis.

As our results show, engineered HIV-specific IgA antibodies are ideal to study the structural effects of IgA on neutralization activity and compartment specific function as neutralizing and arming antibodies. The antibodies of the present invention and the data provided herein support our findings that IgA1 variants of CD4i-specific antibodies (e.g., F425-A1g8) have substantial independent neutralization activity against viral infections (particularly infective agents of the Retroviridae family, e.g., lentiviruses, e.g., HIV-1 and HIV-2) compared to neutralization by the CD4i IgG antibodies known in the art. Our results strongly suggest that the unique molecular structure of IgA antibody variants, particularly CD4i-specific IgA antibodies, can play an important role in virus neutralization and, therefore, therapies for viral infections.

### Antibodies of the Invention

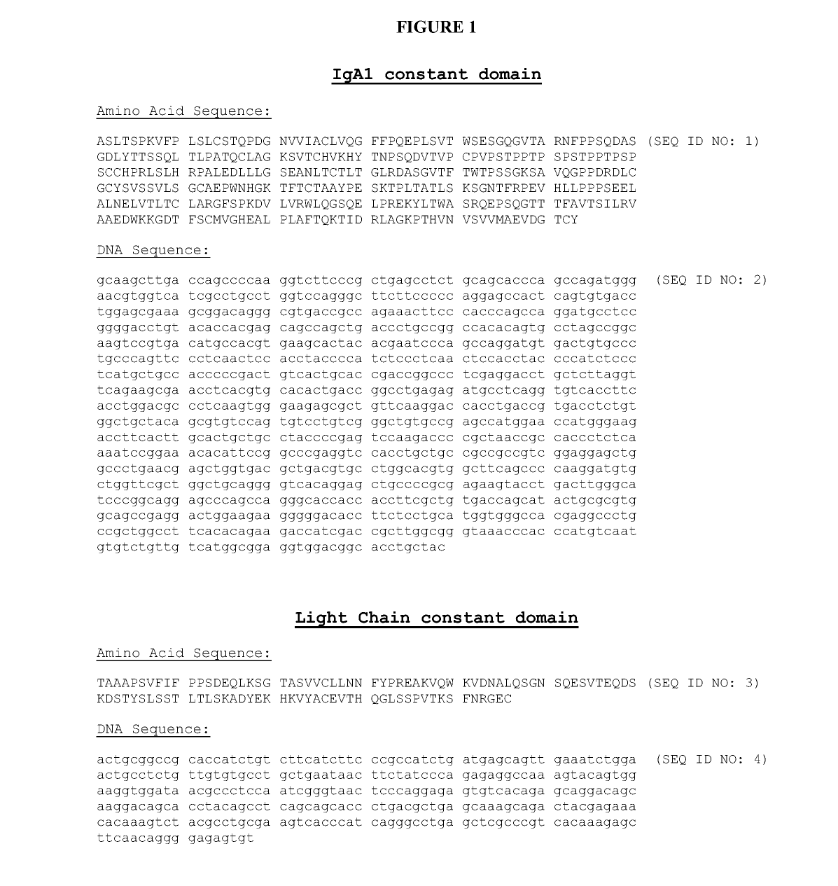

The present invention relates to isolated IgA antibodies, or fragments thereof, having variable domains derived from an antibody that specifically binds to a CD4-induced (CD4i) epitope. The isolated IgA antibody may include at least one heavy chain constant domain of IgA (e.g., CH1, CH2, or CH3), at least two heavy chain constant domains of IgA (e.g., CH1 and CH2, CH1 and CH3, or CH2 and CH3), or all three heavy chain constant domains of IgA (e.g., CH1, CH2, and CH3). In one embodiment, the isolated IgA antibody includes a heavy chain constant region amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 1 (FIG. 1). The isolated IgA antibody may include a light chain constant domain of IgA having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 3 (FIG. 1). In certain embodiments, the antibody, or fragment thereof, is a chimeric antibody containing IgA constant domains and IgG-derived variable domains. The isolated IgA antibody, or fragment thereof, of the invention may have variable domains derived from an IgG antibody, such as the F425-A1g8, 17b, 48d, E51, X5, or m16 antibody.

Isolated IgA antibodies, or fragments thereof, having variable domains derived from a F425-A1g8 antibody may include a heavy chain variable domain and/or a light chain variable domain, wherein the heavy chain variable domain includes a CDR-H1 including the amino acid sequence GFIFSAFV (SEQ ID NO: 9), CDR-H2 including the amino acid sequence VWYDGNSK (SEQ ID NO: 11), and CDR-H3 including the amino acid sequence AREWVADDDTFDGFDV (SEQ ID NO: 13), and/or wherein the light chain variable domain includes a CDR-L1 including the amino acid sequence QSVTNS (SEQ ID NO: 15), CDR-L2 including the amino acid sequence DAS (SEQ ID NO: 17), and CDR-L3 including the amino acid sequence QQRSNWPPEVT (SEQ ID NO: 19). In another embodiment, the isolated IgA antibodies, or fragments thereof, include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 5 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 7.

Isolated IgA antibodies, or fragments thereof, having variable domains derived from a 17b antibody may include a heavy chain variable domain and/or a light chain variable domain, wherein the heavy chain variable domain includes a CDR-H1 including the amino acid sequence GDTFIRYS (SEQ ID NO: 25), CDR-H2 including the amino acid sequence IITILDVT (SEQ ID NO: 27), and CDR-H3 including the amino acid sequence AGVYRGRGGRGEYDNNGFLKH (SEQ ID NO: 29), and/or wherein the light chain variable domain includes a CDR-L1 including the amino acid sequence ESVSSD (SEQ ID NO: 31), CDR-L2 including the amino acid sequence GAS (SEQ ID NO: 33), and CDR-L3 including the amino acid sequence QQYNNWPPRYT (SEQ ID NO: 35). In another embodiment, the isolated IgA antibodies, or fragments thereof, include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 21 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 23.

Isolated IgA antibodies, or fragments thereof, having variable domains derived from a 48d antibody may include a heavy chain variable domain and/or a light chain variable domain, wherein the heavy chain variable domain includes a CDR-H1 including the amino acid sequence GYTFSDFY (SEQ ID NO: 41), CDR-H2 including the amino acid sequence IDPEDADT (SEQ ID NO: 43), and CDR-H3 including the amino acid sequence AADPWELNAFNV (SEQ ID NO: 45), and/or wherein the light chain variable domain includes a CDR-L1 including the amino acid sequence QDISTW (SEQ ID NO: 47), CDR-L2 including the amino acid sequence AAS (SEQ ID NO: 49), and CDR-L3 including the amino acid sequence QQANSFFT (SEQ ID NO: 51). In another embodiment, the isolated IgA antibodies, or fragments thereof, include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 37 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 39.

Isolated IgA antibodies, or fragments thereof, having variable domains derived from a E51 antibody may include a heavy chain variable domain and/or a light chain variable domain, wherein the heavy chain variable domain includes a CDR-H1 including the amino acid sequence GATLNSHA (SEQ ID NO: 57), CDR-H2 including the amino acid sequence IIPIFGSS (SEQ ID NO: 59), and CDR-H3 including the amino acid sequence ASNSIAGVAAAGDYADYDGGYYYDMDV (SEQ ID NO: 61), and/or wherein the light chain variable domain includes a CDR-L1 including the amino acid sequence SSNIGNND (SEQ ID NO: 63), CDR-L2 including the amino acid sequence ENN (SEQ ID NO: 65), and CDR-L3 including the amino acid sequence GTWDSSLSAVV (SEQ ID NO: 67). In another embodiment, the isolated IgA antibodies, or fragments thereof, include a heavy chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 53 and/or a light chain variable domain including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 55.

In some embodiments, the isolated IgA antibodies, or fragments thereof, are associated with a J chain polypeptide including an amino acid sequence having at least about 60%, 65%, 70%, 75%, or 80% sequence identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 69 or 70.

An isolated IgA antibody, or fragment thereof, may be derivatized or linked to another agent, such as a label or a toxin. The IgA antibody can be functionally linked (by chemical coupling, genetic fusion, noncovalent association or otherwise) to one or more other molecular entities. The agent may be a label used for detection purposes, such as a fluorescent compound, an enzyme, a prosthetic group, a luminescent material, a bioluminescent material, or a radioactive material. The labeled antibodies may be used, for example, diagnostically and/or experimentally. In other embodiments, the agent is as toxin such as a cytotoxic agent used, for example, for concurrent treatment therapies.

The isolated IgA antibodies of the invention may be chimeric, human, humanized, or synthetic.

### Therapeutic Uses

The isolated IgA antibodies of the invention having variable domains derived from antibodies that specifically bind to a CD4-induced epitope of a polypeptide (e.g., F425-A1g8) may be used for therapeutic applications. For example, the IgA antibodies can be used for the treatment of a subject (e.g., a human) having a viral infection. The viral infection may be caused by an infective agent, such as an infective agent of the Retroviridae family (e.g., a lentivirus, e.g., HIV, e.g., HIV-1 or HIV-2). In some embodiments, the viral infection may be treated at an early stage, prior to the development of disease symptoms, or alternatively at a later stage in which one or more symptoms of disease have manifested. In other embodiments, the isolated IgA antibodies can be used to prophylactically treat a subject (e.g., a human) having an increased risk of viral infection (e.g., an HIV infection). The IgA CD4i-specific antibodies of the invention can be used to treat any disease or condition for which there exists, or is suspected to potentially later exist, a suitable candidate target (e.g., a viral protein, e.g., gp120, e.g., CD4i).

In addition to therapeutic uses, the isolated IgA antibodies of the invention can be used for other purposes, including diagnostic methods, such as diagnostic methods for any disease or condition for which there is a suitable candidate target of the antibody (e.g., AIDS or an HIV infection).

### Methods of Administration

The CD4i-specific IgA antibodies of the invention will be formulated, dosed, and administered in a fashion consistent with good medical practice. Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and the patient response to the treatment. Additionally, a person having a greater risk of developing a proliferative or pathogenic disease may receive treatment to inhibit or delay the onset of symptoms.

The IgA antibodies of the invention described herein are administered to a subject (e.g., a human) intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularly, mucosally, intrapericardially, intraumbilically, intraocularally, orally, topically, locally, by inhalation, by injection, by infusion, by continuous infusion, by localized perfusion bathing target cells directly, by catheter, by lavage, in cremes, or in lipid compositions, in accord with known methods. Preferably, the isolated IgA antibodies are administered by infusion, by continuous infusion, mucosally, or subcutaneously. Alternatively, it is envisioned that the antibodies may be delivered by gene therapy, especially gene therapy targeted to the mucosa of the subject.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window at the site of release (e.g., the gastro-intestinal tract); or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The compositions of the invention may be administered to provide pre-exposure prophylaxis or after a subject has been exposed to virus (e.g., HIV). The composition may be administered, e.g., 1, 2, 3, 4, 5, 6, 7, 8, 9, 10, 15, 20, 30, 35, 40, 45, 50, 55, or 60 minutes, 2, 4, 6, 10, 15, or 24 hours, 2, 3, 5, or 7 days, 2, 4, 6 or 8 weeks, or even 3, 4, or 6 months pre-exposure, or may be administered to the subject 15-30 minutes or 1, 2, 3, 4, 5, 6, 7, 8, 9, 10, 12, 20, 24, 48, or 72 hours, 2, 3, 5, or 7 days, 2, 4, 6 or 8 weeks, 3, 4, 6, or 9 months, 1, 2, 3, 4, 5, 6, 7, 8, 9, 10, 15, 20 years or longer post-exposure to the infective agent.

When treating disease (e.g., AIDS due to HIV infection), the compositions of the invention may be administered to the subject either before the occurrence of symptoms or a definitive diagnosis or after diagnosis or symptoms become evident. For example, the composition may be administered, e.g., immediately after diagnosis or the clinical recognition of symptoms or 2, 4, 6, 10, 15, or 24 hours, 2, 3, 5, or 7 days, 2, 4, 6 or 8 weeks, 3, 4, or 6 months, or even 2, 4, 6, 8, 10, 15, or 20 or more years after diagnosis or detection of symptoms.

The compositions may be sterilized by conventional sterilization techniques, or may be sterile filtered. The resulting aqueous solutions may be packaged for use as is or lyophilized. The lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the CD4i-specific IgA antibody and, if desired, one or more immunomodulatory agents, such as in a sealed package of tablets or capsules, or in a suitable dry powder inhaler (DPI) capable of administering one or more doses.

Dosages

The dosage administered depends on the subject to be treated (e.g., the age, body weight, capacity of the immune system, and general health of the subject being treated), the form of administration (e.g., as a solid or liquid), the manner of administration (e.g., by injection, inhalation, dry powder propellant), and the cells targeted (e.g., mucosal cells, epithelial cells, such as blood vessel epithelial cells, nasal epithelial cells, or pulmonary epithelial cells). Additionally, pharmacogenomic (the effect of genotype on the pharmacokinetic, pharmacodynamic, or efficacy profile of a therapeutic) information about a particular patient may affect the dosage used. The composition is preferably administered in an amount that provides a sufficient level of the antibody to yield a therapeutic effect in the subject without undue adverse physiological effects caused by treatment.

Single or multiple administrations of the compositions of the present invention may be given (pre- or post-exposure) to a subject (e.g., one administration or administration two or more times). For example, subjects who are particularly susceptible to viral infection (e.g., HW infection) may require multiple treatments to establish and/or maintain protection against the virus. Levels of induced immunity provided by the pharmaceutical compositions described herein can be monitored by, for example, measuring amounts of the neutralizing antibodies or the level of CD4⁺ cells in the subject. The dosages may then be adjusted or repeated as necessary to maintain desired levels of protection against the virus.

Alternatively, the efficacy of treatment can be determined by monitoring the level of the antigenic gene product, or fragment thereof, expressed in a subject (e.g., a human) following administration of the compositions of the invention. For example, the blood or lymph of a subject can be tested for antigenic gene product, or fragment thereof, using, e.g., standard assays known in the art (see, e.g., Human Interferon-Alpha Multi-Species ELISA kit (Product No. 41105) and the Human Interferon-Alpha Serum Sample kit (Product No. 41110) from Pestka Biomedical Laboratories (PBL), Piscataway, N.J.).

A single dose of the compositions of the invention may achieve protection, pre-exposure, from infective agents. In addition, a single dose administered post-exposure to a viral or other infective agent can function as a treatment according to the present invention. Multiple doses (e.g., 2, 3, 4, 5, or more doses) can also be administered, in necessary, to these subjects.

A single dose of the compositions of the invention can also be used to achieve therapy in subjects being treated for a disease. Multiple doses (e.g., 2, 3, 4, 5, or more doses) can also be administered, in necessary, to these subjects.

The appropriate dosage and treatment regimen can be determined by one skilled in the art.

Carriers, Excipients, Diluents

Therapeutic formulations of the compositions of the invention are prepared using standard methods known in the art by mixing the active ingredient having the desired degree of purity with optional physiologically acceptable carriers, excipients, or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

### EXAMPLES

### Example 1

### Materials and Methods

### Monoclonal Antibodies, Virus, and Cell Lines

The neutralizing IgG antibody F425-A1g8 was generated in our laboratory, as previously described (Cavacini et al., AIDS. 17:685-689, 2003), and was shown to bind to the CD4i site of gp120. The immunoglobulin expression vectors pLC-HuCκ, pHC-HuCγ1, and pHC-HuCα1 were obtained which contained the human immunoglobulin light chain, heavy chain γ1, and α1 constant regions, respectively. The CHO-K1 cells were from American Type Culture Collection. The following reagents were obtained through the AIDS Research and Reference Reagent Program, Division of AIDS, NIAID, NIH: SF162 (R5) from Dr. Jay Levy; 89.6 (R5X4) from Dr. Ronald Collman; BaL (R5) from Dr. Suzanne Gartner, Dr. Mikulas Popovic, and Dr. Robert Gallo; 93MW960 (clade C, R5) from Dr. Robert Bollinger and the UNAIDS Network for HIV; JR-FL (R5) from Dr. Irvin Chen; Isolate 67970 (CXCR4) was from Dr. David Montefiori. TZM-bl cells from Dr. John C. Kappes, Dr. Xiaoyun Wu, and Transzyme, Inc.

### Construction, Production, and Purification of IgA F425A1g8 Antibody Variants

F425-A1g8 VH and VL were PCR amplified from a F425-A1g8 hybridoma cell line using specific primers (Table 1) which introduced restriction enzymes sites (5′ NheI and 3′ HindIII for VH; 5′ NheI and 3′ NotI for VL). The VH fragment was cloned separately into the expression vectors pHC-HuCγ1 and pHC-huCα1. The VL was cloned into vector pLC-huCκ. Paired purified plasmids encoding the F425-A1g8 light chain versus IgG1 heavy chain and F425-A1g8 light chain versus IgA1 heavy chain were co-transfected into CHO-K1 cells in equimolar amounts in 6-well plates using lipofectamine LTX reagent (Invitrogen Life Technologies). Selection with G418 (800 μg/ml) and puromycin (10 mg/ml) were added after 24 hours. Cells were plated in 96-well plates with selection, and wells were screened when dense using standard IgG and IgA capture ELISAs. Positive wells were cloned by limiting dilution until a stable producing cell line was isolated. Antibody was purified from culture supernatant using protein L chromatography. Purity was confirmed using SDS-PAGE.

TABLE 1
Primers for amplifying variable domain
of F425-A1g8 and human J chain fragment

| | |
|---|---|
| F425A1g8VH-5′ | CTAGCTAGCCGCCACCATGGAGCTTGG |
| (Nhe I) | (SEQ ID NO: 71) |
| F425A1g8VH-3′ | CCCTTGAAGCTTGCTGAAGAGACGGTG |
| (Hind III) | (SEQ ID NO: 72) |
| F425A1g8VL-5′ | CTAGCTAGCCGCCACCATGGACATGAGG |
| (Nhe I) | (SEQ ID NO: 73) |
| F425A1g8VL-3′ | GACAGATGGTGCGGCCGCAGTTCGTTTGATATCC |
| (Not I) | (SEQ ID NO: 74) |
| Human J Chain-5′ | CTAGCTAGCATGAAGAACCATTTGC |
| (Nhe I) | (SEQ ID NO: 75) |
| Human J Chain-3′ | TGCGATATCTTAGTCAGGATAGCAGG |
| (EcoR V) | (SEQ ID NO: 76) |

Restriction sites are bolded and underlined.
VH: variable domain of heavy chain;
VL: variable domain of light chain;
J: human J chain.

### Immunoreactivity of Recombinant IgA F425A1g8 Antibody Variants

Live cell ELISA assay was performed to determine the immunoreactivity of F425-Alg8 variances to the CD4 binding site. SF2 infected cells (1×10⁶) were incubated with antibody at 20, 10, 5, and 2.5 μg/ml for 30 minutes followed by washing and incubation with HRP-conjugated goat anti-human IgG or IgA (Southern Biotechnology Associates). The human monoclonal antibodies b12 IgG1 or IgA1 were run at 20 μg/ml as a standard to determine relative reactivity of the IgA F425-A1 g8 antibody variants with HIV. After washing, cells were re-suspended in 100 μl TMB substrate and incubated for 10 minutes. Reaction was stopped by adding 100 μl of 1M phosphoric acid and samples were read on a plate reader at 450 nm.

### Direct Viral Neutralization

The neutralization activity of isolated IgA F425-A1g8 antibody variants were determined in vitro using a TZM-bl assay with a panel of three isolates including SF162, JR-FL, and 67970. Primary isolate virus was grown in PHA-stimulated peripheral blood mononuclear cells (PBMC) as previously described (Cavacini et al., AIDS Res. Hum. Retroviruses. 19:785-792; Cavacini et al., AIDS. 17:685-689, 2003; Wei et al., Antimicrob. Agents Chemother. 46:1896-1905, 2002) and titered on TZM-bl cells (Duval et al., J. Virol. 82:4671-4674, 2008) to determine TCID₅₀. Serial two-fold dilutions of IgA F425-A1g8 antibody variants were incubated with virus stock diluted to 100 TCID₅₀ for 1 hour at 37° C. prior to the addition of TZM-bl cells (1×10⁴ c/well). Using β-galactosidase reagent from Promega as an indicator of HIV replication, plates were incubated for 48 hours at 37° C. and 5% CO₂ prior to the measurement of β-galactosidase activity. Percent neutralization was determined based on control wells of virus and media and IC₅₀ and IC₉₀ values calculated by regression curve analysis.

Antibody Dependent Cell-Mediated Viral Inhibition (ADCVI)

ADCVI activity was measured using HIV grown in PHA-stimulated PBMC as previously described (Miranda et al., J. Immunol. 178:7132-7138, 2007). Neutrophils were obtained from peripheral blood of sero-negative donors by Ficoll-Hypaque gradient centrifugation. Antibodies were titered in 96-well, round-bottom plates in 50 μl of media containing 20% heat-inactivated FBS. Target cells were PBMC productively infected with HIV-1 four days prior to use as previously described (Cavacini et al., J. Virol. 73:9638-9641, 1999), and 1×10⁵ infected cells were added per well in 50 μl. Within 10 minutes of the combination of antibody and infected cells, neutrophils were added to the wells at 1×10⁶ effector cells/well in 100 μl, resulting in a effector:target (E:T) ratio of 10:1. After 4 hours, in order to measure the surviving infectious virus, PHA stimulated PBMC were added as indicator cells (1×10⁵/well). These indicator PBMC were incubated for seven days in the presence of IL-2 at which time the supernatant was quantitated for p24 by a p24-specific ELISA (Stubbe et al., J. Immunol. 164:1952-1960, 2000). IC₅₀ values were determined by linear regression analysis and significance was ascertained by student's t-test. Control wells included irrelevant antibody, no effectors, or no targets to determine background release of virus, maximal production of virus, and whether PMN alone were infected, respectively. Viral inhibition was calculated based on the p24 amount from an irrelevant antibody control. Experiments were repeated three to five times.

### Example 2

### Immunoreactivity of F425-A1g8 IgA Antibody Variants

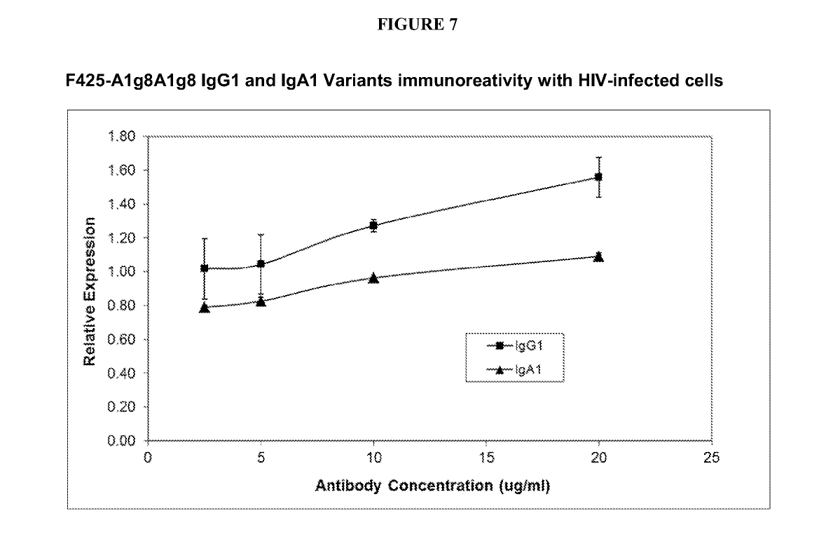

To determine the immunoreactivity of F425-A1g8 antibody variants with the CD4i epitope on HIV infected cells, a live cell ELISA assay was used. Since HRP-conjugated secondary antibodies directly binding to the light chain may be competed by antigen, IgG or IgA isotype-specific secondary antibodies had to be used. Therefore, b12 IgG1 and IgA1 were used to establish relative reactivity by comparing the absorbance (optical density) obtained with F425-A1g8 antibody variants with that obtained from the b12 controls. The results are expressed as a “relative expression” b12 unit (OD F425-A1g8/OD b12). As shown in FIG. 7, the reactivity of F425-A1g8 IgA1 with HIV was retained. In this experiment, SF2-infected cells (1×10⁶) were incubated with titered antibodies of F425-A1g8 IgG1 (square) and IgA1 (triangle) which were detected using HRP-conjugated goat anti-human IgG or IgA. Bound antibody was visualized using TMB substrate and stopped by 100 μl of 1M phosphoric acid. The OD was read on plate reader at 450 nm. b12 IgG1 or IgA1 (20 μg/ml) was used as a standard to determine relative reactivity of the F425-A1g8 variants with HIV. In particular, the IgG1 variant of F425-A1g8 had more relative binding than that observed for the IgA1 variant.

### Example 3

### Neutralizing Activity of F425-A1g8 IgA Antibody Variants Against HIV-1

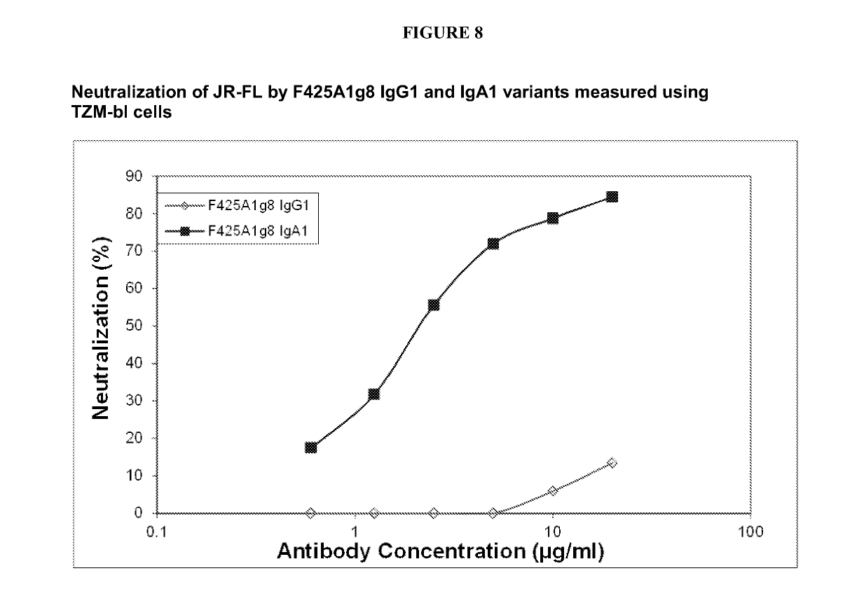

Neutralization of HIV was tested using TZM-bl cells and three clade B primary isolate viruses (SF162, JR-FL, 67970) grown in PBMCs. Serial dilutions of antibody were tested and IC₅₀ values for JR-FL and 67970, and IC₉₀ for SF162 were determined by linear regression. In contrast to minimal neutralization by F425-A1g8 IgG1 in the absence of soluble CD4 (sCD4), the IgA1 variant of the antibody displayed significant neutralization activity against a number of HIV clade B isolates in the absence of sCD4 as shown in Table 2 and FIG. 8. As shown in FIG. 8, the neutralizing activity against JR-FL by the IgA1 antibody variant of F425-A1g8 was significantly increased compared to that of the F425-A1g8 IgG1 antibody. In this experiment, JR-FL (100 TCID₅₀) was incubated with two-fold serial dilutions of F425-A1g8 IgG1 (green diamonds) and IgA1 (blue squares) antibody variants for one hour prior to the addition of TZM-bl cells. HIV was measured as β-galactosidase activity after 48 hours. Percent neutralization was determined by the formula ((control−test)/control)×100.

As shown in Table 2, even though the F425-A1g8 IgG1 antibody neutralized the SF162 isolate, the IgA1 antibody variant of F425-A1g8 displayed significantly increased neutralization activity. These results were the mean of triplicate wells and were representative of at least three independent experiments. This differential neutralization was confirmed in studies using tier 1 and reference panel virus (n=7 including BaL and SF162) grown in 293T cells. Increased neutralization mediated by IgA1 occurs despite relatively decreased immunoreactivity of the IgA1 to SF2 infected cells as compared to the IgG1.

TABLE 2
Neutralization of HIV-1 by F425-Alg8 IgG and IgA antibody variants

| | JR-FL | 67970 | SF162 |
|---|---|---|---|
| | Clade B, R5 | Clade B, X4 | Clade B, R5 |
| | (IC₅₀)^{a} | (IC₅₀) | (IC₉₀)^{b} |
| IgG1^{c} | >40 | >40 | 2.3 ± 1.4 |
| IgA1^{d} | 1.73 ± 0.2 | 23.3 ± 14.3 | 1.7 ± 1.0 |

^{a}and ^{b}IC₅₀ or IC₉₀ concentration (μg/ml) of antibody required for 50% or 90% inhibition of HIV, respectively.
^{c}F425-Alg8 IgG1 antibody variant expressed from CHO-K1 cells.
^{d}F425-Alg8 IgA1 antibody variant expressed from CHO-K1 cells.

### Example 4

### Functional Activity of F425-A1g8 IgA Antibody Variant in Mediating ADCVI

We also investigated the impact of the IgA1 constant domain of the F425-A1g8 IgA antibody variant on functional ability of ADCVI for HIV and HIV-infected cells. HIV-1-binding antibodies mediate ADCVI through an interaction with specific Fc receptors on effector cells, resulting in effector cell-mediated destruction of infected cells with antibody-bound antigen (Forthal et al., J. Immunol. 178:6596-6603, 2007). Therefore, ADCVI would be a useful assay to determine the ability of the isotype variants of specific antibodies to mediate effector cell destruction of or inhibit HIV replication in an infected target cell population in vivo. Polymorphonuclear leukocytes (PMN) or neutrophils are the predominant (60-70%) type of white blood cell in the circulation and play a critical role in innate immunity against infections. PMN consistently express multiple receptors for IgG including FcγRIIa (CD32), FcγRIIIa (CD16), and FcγRIIIb. They also express FcγRI (CD64) following induction with G-CSF. In addition to Fc receptors for IgG, PMN also express Fc receptors for IgA (FcαR, CD89). Cross-linking Fcγ receptors as well as cross-linking of the IgA receptor on PMN by monoclonal antibodies have been shown to be critical to induce ADCC against tumor cells (Hernandez-Ilizaliturri et al., Clin. Cancer Res. 9:5866-5873, 2003; Rafiq et al., J. Clin. Invest. 110:71-79, 2002). Therefore, although traditional ADCVI (or ADCC) assays are based on mononuclear cell populations, we propose to use neutrophils as effectors.

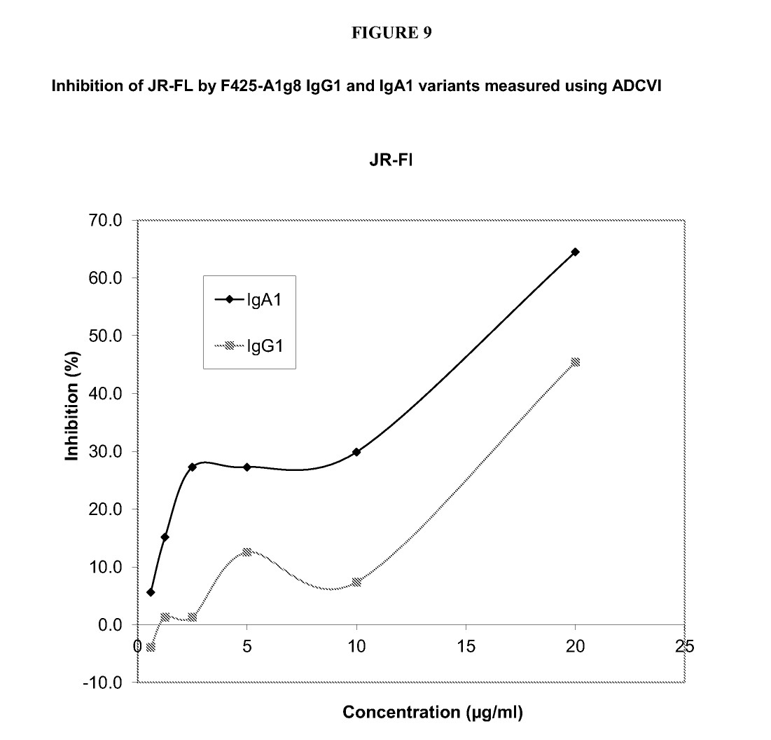

Since the binding of F425-A1g8 was different with strains of virions, a total of five isolates, including clade B representing R5, R5X4, and X4 isolates and clade C isolate (R5), were tested in this variant of the neutralization assay. Antibody-mediated destruction of HIV and HIV-infected cells is determined by testing the inhibition of subsequent HIV replication or p24 levels. The results of these assays are summarized in Table 3 as well as in FIG. 9, which depicts data specifically of the tested JR-FL strain. As exemplified in FIG. 9, the F425-A1g8 IgA1 antibody variant showed more robust ADCVI activity for clade B isolate JR-FL compared to that of the IgG1 antibody variant. For these experiments, F425-A1g8 variants were incubated with virus-infected (e.g., JR-FL infected) PBMC just prior to adding neutrophils at an E:T ratio of 10:1. After 4 hours, PHA stimulated PBMC were added as indicator cells and p24 was quantitated by ELISA after one week. Percent inhibition was determined by the formula: [(p24 control−p24 test)/p24 control]×100.

As shown in Table 3, the F425-A1g8 IgA1 antibody variant showed significant ADCVI activity for both clade B isolates and a single clade C isolate. For two of four clade B isolates (SF162 and JR-FL, both R5), F425A1g8 IgG1 failed to mediate ADCVI activity whereas significant activity was observed for the F425-A1g8 A1 antibody variant with p-values ranging from 0.0008-0.05 for multiple experiments. Two clade B strains, BaL (R5) and 89.6 (R5X4) failed to be inhibited by either isotype variant at the concentrations tested. Both antibody isotype variants inhibited the clade C isolate, 93MW960. The IgG1 isotype had greater activity against the Clade C isolate than IgA1 (p-value from 0.0012 to 0.0598).

The variant in impact of isotype in ADCVI may result from affinity and/or binding specificity of the Fc fragment of the IgA1 subclass (compared to the IgG1 subclass) with Fc receptors on the surface of neutrophils. On the other hand, the antigen density and epitope orientation may result in differences in outcome. There was no viral inhibition in mock control wells which contained antibody, target cells, or indicator cells without neutrophils. Viral replication was similar for control wells containing effector cells, target cells without antibody, and target cells alone.

TABLE 3
ADCVI activity of HIV-1 by F425Alg8 IgA1 and IgG1
antibody variants

| | IC₅₀ (μg/ml)^{a} | | | | |
|---|---|---|---|---|---|
| | BaL | JR-FL | | | |
| | Clade | Clade | 93MW960 | 89.6 | SF162 |
| | B, R5 | B, R5 | Clade C R5 | Clade B, R5X4 | Clade B, R5 |
| | (n = 6) | (n = 6) | (n = 5) | (n = 3) | (n = 3) |
| IgG1 | >40 | >40 | 9.5 ± 7.9 | >40 | >40 |
| IgA1 | >40 | 16.6 ± 5.1 | 18.3 ± 13.4 | >40 | 6.1 ± 5.9 |

^{a}The ADCVI activity was determined by IC₅₀ that represents concentration (μg/ml) of antibody required for 50% inhibition of HIV.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

What is claimed is:

1. An isolated IgA antibody, or fragment thereof, wherein the variable domains of the IgA antibody are derived from an antibody that specifically binds to a CD4-induced (CD4i) epitope of a polypeptide.

2. The isolated IgA antibody of claim 1, wherein the IgA antibody comprises at least one heavy chain constant domain of IgA selected from the group consisting of a CH1, CH2, and CH3 constant domain of IgA.

3. The isolated IgA antibody of claim 2, wherein the IgA antibody comprises at least two heavy chain constant domains of IgA selected from the group consisting of a CH1, CH2, and CH3 constant domain of IgA.

4. The isolated IgA antibody of claim 3, wherein the IgA antibody comprises three heavy chain constant domains of IgA, wherein the three heavy chain constant domains are CH1, CH2, and CH3 constant domains of IgA.

5. The isolated IgA antibody of claim 4, wherein the IgA antibody comprises a heavy chain constant region amino acid sequence having at least 80% sequence identity to SEQ ID NO: 1.

6. The isolated IgA antibody of any one of claims 1-5, wherein the IgA antibody comprises a light chain constant domain of IgA.

7. The isolated IgA antibody of claim 6, wherein the IgA antibody comprises a light chain constant domain amino acid sequence having at least 80% sequence identity to SEQ ID NO: 3.

8. The isolated IgA antibody of any one of claim 1-7, wherein the antibody that specifically binds to a CD4i epitope of a polypeptide is selected from the group consisting of F425-A1g8, 17b, 48d, E51, X5, and m16.

9. The isolated IgA antibody of claim 8, wherein the IgA antibody comprises a heavy chain variable domain and a light chain variable domain, wherein the heavy chain variable domain comprises a CDR-H1, CDR-H2, and CDR-H3, wherein:
(a) CDR-H1 comprises the amino acid sequence GFIFSAFV (SEQ ID NO: 9);
(b) CDR-H2 comprises the amino acid sequence VWYDGNSK (SEQ ID NO: 11); and
(c) CDR-H3 comprises the amino acid sequence AREWVADDDTFDGFDV (SEQ ID NO: 13);
and wherein the light chain variable domain comprises a CDR-L1, CDR-L2, and CDR-L3, wherein:
(d) CDR-L1 comprises the amino acid sequence QSVTNS (SEQ ID NO: 15);
(e) CDR-L2 comprises the amino acid sequence DAS (SEQ ID NO: 17); and
(f) CDR-L3 comprises the amino acid sequence QQRSNWPPEVT (SEQ ID NO: 19).

10. The isolated IgA antibody of claim 9, wherein the heavy chain variable domain comprises an amino acid sequence having at least 80% sequence identity to SEQ ID NO: 5, and wherein the light chain variable domain comprises an amino acid sequence having at least 80% sequence identity to SEQ ID NO: 7.

11. The isolated IgA antibody of claim 8, wherein the IgA antibody comprises a heavy chain variable domain and a light chain variable domain, wherein the heavy chain variable domain comprises a CDR-H1, CDR-H2, and CDR-H3, wherein:
(a) CDR-H1 comprises the amino acid sequence GDTFIRYS (SEQ ID NO: 25);
(b) CDR-H2 comprises the amino acid sequence IITILDVT (SEQ ID NO: 27); and
(c) CDR-H3 comprises the amino acid sequence AGVYRGRGGRGEYDNNGFLKH (SEQ ID NO: 29);
and wherein the light chain variable domain comprises a CDR-L1, CDR-L2, and CDR-L3, wherein:
(d) CDR-L1 comprises the amino acid sequence ESVSSD (SEQ ID NO: 31);
(e) CDR-L2 comprises the amino acid sequence GAS (SEQ ID NO: 33); and
(f) CDR-L3 comprises the amino acid sequence QQYNNWPPRYT (SEQ ID NO: 35).

12. The isolated IgA antibody of claim 11, wherein the heavy chain variable domain comprises an amino acid sequence having at least 80% sequence identity to SEQ ID NO: 21, and wherein the light chain variable domain comprises an amino acid sequence having at least 80% sequence identity to SEQ ID NO: 23.

13. The isolated IgA antibody of claim 8, wherein the IgA antibody comprises a heavy chain variable domain and a light chain variable domain, wherein the heavy chain variable domain comprises a CDR-H1, CDR-H2, and CDR-H3, wherein:
(a) CDR-H1 comprises the amino acid sequence GYTFSDFY (SEQ ID NO: 41);
(b) CDR-H2 comprises the amino acid sequence IDPEDADT (SEQ ID NO: 43); and
(c) CDR-H3 comprises the amino acid sequence AADPWELNAFNV (SEQ ID NO: 45);
and wherein the light chain variable domain comprises a CDR-L1, CDR-L2, and CDR-L3, wherein:
(d) CDR-L1 comprises the amino acid sequence QDISTW (SEQ ID NO: 47);
(e) CDR-L2 comprises the amino acid sequence AAS (SEQ ID NO: 49); and
(f) CDR-L3 comprises the amino acid sequence QQANSFFT (SEQ ID NO: 51).

14. The isolated IgA antibody of claim 13, wherein the heavy chain variable domain comprises an amino acid sequence having at least 80% sequence identity to SEQ ID NO: 37, and wherein the light chain variable domain comprises an amino acid sequence having at least 80% sequence identity to SEQ ID NO: 39.

15. The isolated IgA antibody of claim 8, wherein the IgA antibody comprises a heavy chain variable domain and a light chain variable domain, wherein the heavy chain variable domain comprises a CDR-H1, CDR-H2, and CDR-H3, wherein:
(a) CDR-H1 comprises the amino acid sequence GATLNSHA (SEQ ID NO: 57);
(b) CDR-H2 comprises the amino acid sequence IIPIFGSS (SEQ ID NO: 59); and
(c) CDR-H3 comprises the amino acid sequence ASNSIAGVAAAGDYADYDGGYYYDMDV (SEQ ID NO: 61);
and wherein the light chain variable domain comprises a CDR-L1, CDR-L2, and CDR-L3, wherein:
(d) CDR-L1 comprises the amino acid sequence SSNIGNND (SEQ ID NO: 63);
(e) CDR-L2 comprises the amino acid sequence ENN (SEQ ID NO: 65); and
(f) CDR-L3 comprises the amino acid sequence GTWDSSLSAVV (SEQ ID NO: 67).

16. The isolated IgA antibody of claim 15, wherein the heavy chain variable domain comprises an amino acid sequence having at least 80% sequence identity to SEQ ID NO: 53, and wherein the light chain variable domain comprises an amino acid sequence having at least 80% sequence identity to SEQ ID NO: 55.

17. The isolated IgA antibody of any one of claims 1-16, wherein the IgA antibody is associated with a J chain polypeptide, wherein the J chain polypeptide comprises an amino acid sequence having at least 80% sequence identity to SEQ ID NO: 69 or 70.

18. The isolated IgA antibody of any one of claims 1-17, wherein the IgA antibody further comprises a label or a toxin.

19. The isolated IgA antibody of any one of claims 1-18, wherein the IgA antibody is chimeric, human, humanized, or synthetic.

20. An isolated IgA antibody, or fragment thereof, that competes for CD4i binding with the IgA antibody, or fragment thereof, of any one of claims 1-19.

21. A method of treating a subject having a viral infection comprising administering a therapeutically effective amount of the isolated IgA antibody of any one of claims 1-20 to the subject, thereby treating the subject.

22. A method of prophylactically treating a subject having an increased risk of a viral infection comprising administering a therapeutically effective amount of the isolated IgA antibody of any one of claims 1-20 to the subject, thereby treating the subject.

23. The method of claim 21 or 22, wherein the viral infection is caused by an infective agent of the Retroviridae family.

24. The method of claim 23, wherein the infective agent is a lentivirus.

25. The method of claim 24, wherein the lentivirus is a human immunodeficiency virus type 1 (HIV-1), human immunodeficiency virus type 2 (HIV-2), simian immunodeficiency virus (SIV), bovine immunodeficiency virus (BIV), feline immunodeficiency virus (FIV), Jembrana disease virus, equine infectious anaemia (EIA), puma lentivirus (PLV), lion lentivirus (LLV), caprine arthritis encephalitis virus (CAEV), or Maedi-Visna virus.

26. The method of claim 25, wherein the lentivirus is a human immunodeficiency virus type 1 (HIV-1).

27. The method of claim 26, wherein the HIV-1 is a clade B HIV-1.

28. The method of claim 26, wherein the HIV-1 is a clade C HIV-1.

29. The method of claim 25, wherein the lentivirus is a human immunodeficiency virus type 2 (HIV-2).

30. The method of any one of claims 21-29, wherein the isolated IgA antibody preferably neutralizes an infective agent.

31. The method of any one of claims 21-30, wherein the subject is human.

32. The method of any one of claims 21-31, wherein the isolated IgA antibody is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularly, mucosally, intrapericardially, intraumbilically, intraocularally, orally, topically, locally, by inhalation, by injection, by infusion, by continuous infusion, by localized perfusion bathing target cells directly, by catheter, by lavage, in cremes, or in lipid compositions.

33. The method of claim 32, wherein the isolated IgA antibody is administered by infusion, by continuous infusion, mucosally, or subcutaneously.

34. The method of any one of claims 21-33, wherein the isolated IgA antibody is administered as a pharmaceutical composition comprising a pharmaceutically acceptable carrier.

35. A polynucleotide encoding a heavy chain and/or light chain of an isolated IgA antibody of any one of claims 1-20.

36. A vector comprising the polynucleotide of claim 35.

37. A host cell comprising the vector of claim 36.

38. The host cell of claim 37, wherein the host cell is a mammalian cell.

39. The host cell of claim 38, wherein the mammalian cell is a CHO cell.

40. A method of producing the isolated IgA antibody of any one of claims 1-20, the method comprising culturing a host cell that comprises a vector of claim 36 in a culture medium.

41. The method of claim 40, wherein the method further comprises recovering the isolated IgA antibody from the host cell or the culture medium.
